SEQUENCE LISTING (25 sequences)

SEQ ID NO: 1 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 2 - protein, 130 aa, Homo sapiens
SGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 3 - protein, 80 aa, Homo sapiens
MWTLGRRAVAGLLASPSPAQAQTLTRVPRPAELAPLCGRRGLRTDIDATCTPRRASSNQRGLNQIWNVKKQSVYLMNLRK

SEQ ID NO: 4 - protein, 11 aa, Artificial Sequence
  source: /note=“Description of Artificial Sequence Synthetic peptide”
YGRKKRRQRRR

SEQ ID NO: 5 - protein, 12 aa, Artificial Sequence
  source: /note=“Description of Artificial Sequence Synthetic peptide”
MYGRKKRRQRRR

SEQ ID NO: 6 - protein, 30 aa, Artificial Sequence
  source: /note=“Description of Artificial Sequence Synthetic polypeptide”
GWTLNSAGYLLGPHAVGNHRSFSDKNGLTS

SEQ ID NO: 7 - protein, 14 aa, Artificial Sequence
  source: /note=“Description of Artificial Sequence Synthetic peptide”
INLKALAALAKKIL

SEQ ID NO: 8 - protein, 27 aa, Artificial Sequence
  source: /note=“Description of Artificial Sequence Synthetic peptide”
GWTLNSAGYLLGKINLKALAALAKKIL

SEQ ID NO: 9 - protein, 18 aa, Artificial Sequence
  source: /note=“Description of Artificial Sequence Synthetic peptide”
RQIKIWFQNRRMKWKKGG

SEQ ID NO: 10 - protein, 16 aa, Artificial Sequence
  source: /note=“Description of Artificial Sequence Synthetic peptide”
RQIKIWFQNRRMKWKK

SEQ ID NO: 11 - protein, 9 aa, Artificial Sequence
  source: /note=“Description of Artificial Sequence Synthetic peptide”
RRRRRRRRR

SEQ ID NO: 12 - protein, 34 aa, Artificial Sequence
  source: /note=“Description of Artificial Sequence Synthetic polypeptide”
DAATATRGRSAASRPTERPRAPARSASRPRRPVE

SEQ ID NO: 13 - protein, 34 aa, Artificial Sequence
  source: /note=“Description of Artificial Sequence Synthetic polypeptide”
NAATATRGRSAASRPTQRPRAPARSASRPRRPVQ

SEQ ID NO: 14 - protein, 224 aa, Artificial Sequence
  source: /note=“Description of Artificial Sequence Synthetic polypeptide”
MYGRKKRRQRRRGGMWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 15 - protein, 16 aa, Artificial Sequence
  source: /note=“Description of Artificial Sequence Synthetic peptide”
GGGGSLVPRGSGGGGS

SEQ ID NO: 16 - protein, 6 aa, Artificial Sequence
  source: /note=“Description of Artificial Sequence Synthetic peptide”
GSGSGS

SEQ ID NO: 17 - protein, 8 aa, Artificial Sequence
  source: /note=“Description of Artificial Sequence Synthetic peptide”
GSGSGSGS

SEQ ID NO: 18 - protein, 11 aa, Artificial Sequence
  source: /note=“Description of Artificial Sequence Synthetic peptide”
GGSGGHMGSGG

SEQ ID NO: 19 - protein, 11 aa, Artificial Sequence
  source: /note=“Description of Artificial Sequence Synthetic peptide”
GGSGGSGGSGG

SEQ ID NO: 20 - protein, 5 aa, Artificial Sequence
  source: /note=“Description of Artificial Sequence Synthetic peptide”
GGSGG

SEQ ID NO: 21 - protein, 18 aa, Artificial Sequence
  source: /note=“Description of Artificial Sequence Synthetic peptide”
GGGSEGGGSEGGGSEGGG

SEQ ID NO: 22 - protein, 8 aa, Artificial Sequence
  source: /note=“Description of Artificial Sequence Synthetic peptide”
AAGAATAA

SEQ ID NO: 23 - protein, 5 aa, Artificial Sequence
  source: /note=“Description of Artificial Sequence Synthetic peptide”
GGGGG

SEQ ID NO: 24 - protein, 5 aa, Artificial Sequence
  source: /note=“Description of Artificial Sequence Synthetic peptide”
GGSSG

SEQ ID NO: 25 - protein, 11 aa, Artificial Sequence
  source: /note=“Description of Artificial Sequence Synthetic peptide”
GSGGGTGGGSG

(19) United States
(12) Patent Application Publication
(10) Pub. No.: US 2022/0378869 A1
(43) Pub. Date: Dec. 1, 2022

(54) USE OF FRATAXIN FOR TREATING LEIGH SYNDROME, FRENCH CANADIAN TYPE

(71) Applicant: Larimar Therapeutics, Inc., Bala Cynwyd (US)

(72) Inventor: Joan David Bettoun, Elkins Park, PA (US)

(21) Appl. No.: 17/627,638

(22) PCT Filed: Jul. 17, 2020
(86) PCT No.: PCT/US2020/042683

§ 371 Date: Jan. 14, 2022

Related U.S. Application Data
(60) Provisional application No. 63/018,498, filed on Apr. 30, 2020.
(60) Provisional application No. 62/875,937, filed on Jul. 18, 2019.

(51) Int. Cl. A61K 38/17; A61P 25/28

(52) U.S. Cl. CPC A61K 38/1709; A61P 25/28

(57) ABSTRACT

The present disclosure provides a method for treatment of a subject suffering from Leigh Syndrome French Canadian Type (LSFC), the method comprising administering to the subject a therapeutically effective amount of a frataxin (FXN) therapeutic compound.

### RELATED APPLICATIONS

This application claims priority to U.S. Provisional Patent Application No. 62/875,937, filed on Jul. 18, 2019 and U.S. Provisional Patent Application No. 63/018,498, filed on Apr. 30, 2020. The entire contents of each of the foregoing applications are hereby incorporated herein by reference.

### SEQUENCE LISTING

The instant application contains a Sequence Listing which has been submitted electronically in ASCII format and is hereby incorporated by reference in its entirety. Said ASCII copy, created on Jul. 17, 2020, is named 130197-00620_SL.txt and is 12,418 bytes in size.

### BACKGROUND

The Leigh Syndrome French Canadian type (LSFC) is an inherited metabolic disorder of clinical onset at the age of between 0-8 months, in which affected children present neurological disorder, mild regression of psychomotor skills, hypotonia, congenital cardiomyopathy and other congenital malformations, cognitive and speech impairment, energy deficit and deadly lactic acidosis episodes, leading to death between 3 and 10 years of age. LSFC was originally reported in patients from the Saguenay-Lac St-Jean region of Quebec, with an incidence of 1 in 2,000 affected births in that region, while the general incidence outside of Quebec is 1 in 40,000.

The general hallmark of the intracellular phenotype for Leigh Syndrome is characterized by a cytochrome C oxidase (COX) deficiency, leading to congenital chronic lactic acidosis, detectable in serum and cerebro-spinal fluid (CSF). In LSFC, a complex IV COX deficiency is detectable in muscle, brain and liver tissues (Debray, F. et al. (2011) J Medical Genetics, 48:183-189; and citations therein).

Genetically LSFC is an autosomal recessive disorder, which has been found associated with mutations in the LRPPRC (Leucine Rich Pentatricopeptide Repeat Containing) gene, which maps to chromosome 2p21. The Monarch Initiative (https://monarchinitiative.org/) lists 74 variants of the LRPPRC gene. The most well-known mutations in the LRPPRC gene described in connection with LSFC are the c.1119C>T mutation resulting in an Ala354Val substitution in the LRPPRC protein, and the C1277Xdel8 deletion, seen in a smaller patient population. Both mutations seem to affect protein stability.

The LRPPRC gene encodes a protein 1343 amino acids long (MW˜130 kDa), primarily mitochondrial, involved in the translation and/or stability of the mitochondrial DNA (mtDNA)-encoded transcripts (Ruzzenente, B. et al. (2012) EMBO J. 31:443-456). LRPPRC protein belongs to the pentatricopeptide repeat (PPR) protein family, whose members are characterized by a repeated motif of 35 amino acids and implicated in RNA binding (Small and Peeters (2000) Protein Sequence Motifs, 25(2): P45-47). LRPPRC protein has been described to function as a complex with the protein SLIRP (stem-loop interacting RNA binding protein), which is involved in the maintenance of mitochondrial (mt) RNAs (Iommarini, L. et al. (2015) Mitochondrion. 22: 96-118).

Currently, there is no FDA-approved treatment for LSFC. Accordingly, there remains a need in the art for effective treatments for LSFC.

### SUMMARY

Protein replacement or supplementation therapy is a well-established approach to metabolic diseases, such as diabetes, lysosomal storage disorders and hemophilia. Frataxin (FXN) is a mitochondrial protein which is deficient in the autosomal recessive disorder Friedreich's Ataxia. The present invention provides a treatment for LSFC by tackling mitochondrial function, making use of Frataxin supplementation therapy.

Accordingly, in one aspect, the present disclosure provides a method of treating Leigh Syndrome, French Canadian Type (LSFC), the method comprising administering to a subject in need thereof an effective amount of a frataxin (FXN) therapeutic compound, such that the LSFC in the subject is treated.

In one aspect, the present disclosure also provides a method of modulating LRPPRC in a subject affected by Leigh Syndrome, French Canadian Type (LSFC), the method comprising administering to the subject an effective amount of a frataxin (FXN) therapeutic compound, such that the LRPPRC in the subject is modulated.

In one aspect, the present disclosure also provides a method of modulating SLIRP in a subject affected by Leigh Syndrome, French Canadian Type (LSFC), the method comprising administering to the subject an effective amount of a frataxin (FXN) therapeutic compound, such that the SLIRP in the subject is modulated.

In some embodiments, the level of at least one downstream target of FXN and/or LRPPRC is modulated in the subject.

In some embodiments, the downstream target of FXN and/or LRPPRC is selected from the group consisting of ADAMTS1, ATF3, CYR61, NR4a1, EGR1, EGR2, EGR3, MAOA and IFN1.

In some embodiments, mitochondrial RNA is stabilized in the subject.

In one aspect, the present disclosure provides a method of modulating LRPPRC in a cell, the method comprising contacting the cell with an effective amount of a frataxin (FXN) therapeutic compound, such that the LRPPRC in the cell is modulated.

In one aspect, the present disclosure also provides a method of modulating SLIRP in a cell, the method comprising contacting the cell with an effective amount of a frataxin (FXN) therapeutic compound, such that the SLIRP in the cell is modulated.

In some embodiments, the level of at least one downstream target of FXN and/or LRPPRC is modulated in the cell.

In some embodiments, the downstream target of FXN and/or LRPPRC is selected from the group consisting of ADAMTS1, ATF3, CYR61, NR4a1, EGR1, EGR2, EGR3, MAOA and IFN1.

In some embodiments, the cell is in a subject. In some embodiments, the subject is affected by LSFC.

In some embodiments, the frataxin therapeutic compound comprises a polypeptide comprising frataxin, or a fragment, variant or derivative thereof. In some embodiments, the frataxin therapeutic compound comprises a nucleic acid sequence encoding a polypeptide comprising frataxin, or a variant, fragment or derivative thereof.

In some embodiments, the frataxin therapeutic compound comprises a fusion protein comprising frataxin, or a variant, fragment or derivative thereof, and an at least one different amino acid sequence.

In some embodiments, the fusion protein comprises an amino acid sequence with at least about 85% sequence identity to SEQ ID NO: 1 or SEQ ID NO: 2, or a fragment or derivative thereof. In some embodiments, the fusion protein comprises an amino acid sequence with at least about 90%, at least about 95% or at least about 99% sequence identity to SEQ ID NO: 1 or SEQ ID NO: 2, or a fragment or derivative thereof. In some embodiments, the fusion protein comprises an amino acid sequence of SEQ ID NO: 1, SEQ ID NO: 2, or a fragment or derivative thereof.

In some embodiments, the at least one different amino acid sequence comprises a cell penetrating peptide (CPP). In some embodiments, the CPP comprises a peptide selected from the group of CPPs listed in the Database of Cell-Penetrating Peptides CPPsite 2.0, or a variant, fragment or derivative thereof.

In some embodiments, the CPP comprises, or consists of, an amino acid sequence with at least about 85%, at least about 90%, at least about 95% or at least about 99% sequence identity to any one of SEQ ID NOs. 4-13, or a fragment or derivative thereof. In some embodiments, the CPP comprises, or consists of, an amino acid sequence with at least about 85%, at least about 90%, at least about 95% or at least about 99% sequence identity to the transduction domain of HIV-TAT (SEQ ID NO: 4), or a fragment or derivative thereof. In some embodiments, the CPP comprises, or consists of, the amino acid sequence of the transduction domain of HIV-TAT (SEQ ID NO: 4), or a fragment or derivative thereof.

In some embodiments, the frataxin therapeutic compound comprises, or consists of, a fusion protein comprising an amino acid sequence with at least about 85% sequence identity to SEQ ID NO: 14, or a fragment or derivative thereof In some embodiments, the frataxin therapeutic compound comprises, or consists of, a fusion protein comprising an amino acid sequence with at least about 90%, at least about 95% or at least about 99% sequence identity to SEQ ID NO: 14, or a fragment or derivative thereof In some embodiments, the frataxin therapeutic compound comprises, or consists of, a fusion protein comprising the amino acid sequence of SEQ ID NO: 14, or a fragment or derivative thereof.

In one aspect, the present disclosure also provides a method of treating Leigh Syndrome, French Canadian Type (LSFC), the method comprising administering to a subject in need thereof an effective amount of a frataxin (FXN) therapeutic compound, wherein the frataxin therapeutic compound comprises, or consists of, a fusion protein comprising the amino acid sequence of SEQ ID NO: 14, or a variant, fragment or derivative thereof, such that the LSFC in the subject is treated.

In some embodiments, the present disclosure also provides a method for evaluating effectiveness of frataxin (FXN) supplementation therapy in a subject with LSFC, the method comprising:

(a) determining a level of CYR61 in a sample from the subject following treatment with FXN supplementation therapy;

(b) comparing the level of CYR61 in (a) with a baseline CYR61 level; and

(c) using the comparison in (b) to determine effectiveness of the FXN supplementation therapy in the subject.

In some embodiments, the method further comprises determining a baseline CYR61 level in a sample from a subject with LSFC obtained prior to administration of the FXN supplementation therapy. In some embodiments, determining a level of CYR61 comprises determining the level of CYR61 protein in the sample. In some embodiments, determining a level of CYR61 comprises determining the level of CYR61 mRNA in the sample.

In one aspect, the present disclosure also provides a method of detecting CYR61 in a biological sample from a subject with Leigh Syndrome, French Canadian Type (LSFC), comprising contacting the biological sample, or a portion thereof, with one or more detection reagents specific for detection of CYR61.

In some embodiments, the subject is being treated with a frataxin supplementation therapy.

In some embodiments, the frataxin supplementation therapy comprises treating the subject with a frataxin fusion protein. In some embodiments, the fusion protein comprises, or consists of, an amino acid sequence with at least about 85% sequence identity to SEQ ID NO: 14, or a fragment or derivative thereof. In some embodiments, the fusion protein comprises, or consists of, an amino acid sequence with at least about 90%, at least about 95% or at least about 99% sequence identity to SEQ ID NO: 14, or a fragment or derivative thereof In some embodiments, the fusion protein comprises, or consists of, the amino acid sequence of SEQ ID NO: 14, or a fragment or derivative thereof.

In one aspect, the present disclosure also provides a method of treating lactic acidosis in a subject with Leigh Syndrome, French Canadian Type (LSFC), the method comprising administering to the subject an effective amount of a frataxin (FXN) therapeutic compound, such that the lactic acidosis in the subject is treated.

In some embodiments, the frataxin therapeutic compound comprises a polypeptide comprising frataxin, or a fragment, variant or derivative thereof. In some embodiments, the frataxin therapeutic compound comprises a nucleic acid sequence encoding a polypeptide comprising frataxin, or a variant, fragment or derivative thereof.

In some embodiments, the frataxin therapeutic compound comprises, or consists of, a fusion protein comprising frataxin, or a variant, fragment or derivative thereof, and an at least one different amino acid sequence. In some embodiments, the fusion protein comprises an amino acid sequence with at least about 85% sequence identity to SEQ ID NO: 1 or SEQ ID NO: 2, or a fragment or derivative thereof. In some embodiments, the fusion protein comprises an amino acid sequence with at least about 90%, at least about 95% or at least about 99% sequence identity to SEQ ID NO: 1 or SEQ ID NO: 2, or a fragment or derivative thereof. In some embodiments, the fusion protein comprises an amino acid sequence of SEQ ID NO: 1, SEQ ID NO: 2, or a fragment or derivative thereof.

In some embodiments, the at least one different amino acid sequence comprises a cell penetrating peptide (CPP). In some embodiments, the CPP comprises a peptide selected from the group of CPPs listed in the Database of Cell-Penetrating Peptides CPPsite 2.0, or a variant, fragment or derivative thereof.

In some embodiments, the CPP comprises, or consists of, an amino acid sequence with at least about 85%, at least about 90%, at least about 95% or at least about 99% sequence identity to any one of SEQ ID NOs. 4-13, or a fragment or derivative thereof. In some embodiments, the CPP comprises, or consists of, an amino acid sequence with at least about 85%, at least about 90%, at least about 95% or at least about 99% sequence identity to the transduction domain of HIV-TAT (SEQ ID NO: 4), or a fragment or derivative thereof. In some embodiments, the CPP comprises, or consists of, the amino acid sequence of the transduction domain of HIV-TAT (SEQ ID NO: 4), or a fragment or derivative thereof.

In some embodiments, the frataxin therapeutic compound comprises, or consists of, a fusion protein comprising an amino acid sequence with at least about 85% sequence identity to SEQ ID NO: 14, or a fragment or derivative thereof In some embodiments, the frataxin therapeutic compound comprises, or consists of, a fusion protein comprising an amino acid sequence with at least about 90%, at least about 95% or at least about 99% sequence identity to SEQ ID NO: 14, or a fragment or derivative thereof In some embodiments, the frataxin therapeutic compound comprises, or consists of, a fusion protein comprising the amino acid sequence of SEQ ID NO: 14, or a fragment or derivative thereof.

In one aspect, the present disclosure provides a method of treating lactic acidosis in a subject with Leigh Syndrome, French Canadian Type (LSFC), the method comprising administering to the subject an effective amount of a frataxin (FXN) therapeutic compound,

wherein the frataxin therapeutic compound comprises a fusion protein comprising, or consisting of, the amino acid sequence of SEQ ID NO: 14, or a variant, fragment or derivative thereof, such that the lactic acidosis in the subject is treated.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale.

FIG. 1 shows the results of analysis of cells transfected with either a control short hairpin (sh) RNA that is not specific for LRPPRC (Scramble-5 Clone, Scr-5) or with shRNA that is specific for LRPPRC (shRNA-LRPPRC; clones KD-1C, KD-2C, KD-3C, KD-4C and KD-21C). FIG. 1, Panel A is a photo of a representative Western blot showing LRPPRC and mitochondrial Cytochrome Oxidase II (mt-0O2) protein levels, and β-actin as an internal control, in cells transfected with Scr-5, or with shRNA-LRPPRC, KD-1C, KD-2C, KD-3C, KD-4C and KD-21C, in accordance with an embodiment of the disclosure. FIG. 1, Panel B is a graph showing the quantification of the expression level (in percentage) of LRPPRC protein and mtCO2 in the control and shRNA transfected cells, in accordance with an embodiment of the disclosure.

FIG. 2 is a graph showing quantification of lactate levels in conditioned media from control cells (Scr-5) or LRPPRC KD cells.

FIG. 3 shows a photograph of the Western blot analysis of cells transfected with either a control short hairpin (sh) RNA that is not specific for frataxin (Scramble) or with shRNA that is specific for frataxin (FXN; Clones A2 and A6). The table below the photograph shows quantification of frataxin levels in the control, A2 and A6 clones.

FIG. 4 is a graph showing the results of a quantitative (Q) RT-PCR analysis of transcripts modulated upon knock down of the LRPPRC transcript. Control Scramble is represented by a closed circle (•), LRPPRC KD clone 2C is represented by an up arrow (↑), and LRPPRC KD clone 21C is represented by a down arrow (↓).

FIG. 5 is a graph showing the results of a quantitative (Q) RT-PCR analysis of transcripts modulated upon knock down of the frataxin transcript. Control Scramble is represented by a closed circle (•), FXN KD clone A2 is represented by a closed square (▪), and FXN KD clone A6 is represented by a down arrow (↓).

FIG. 6 shows the amount CYR61 protein secreted into the media of hFXN-KD and LRPPCR-KD cells. Specifically, FIG. 6, Panel A is a bar graph showing the amount of CYR61 protein in the media from hFXN-KD and control (scramble) cells treated with vehicle or a TAT-hFXN fusion protein. FIG. 6, Panel B is a bar graph showing the amount of CYR61 protein in the media from LRPPRC-KD and control (scramble) cells treated with vehicle or different concentrations of a TAT-hFXN fusion protein. FIG. 6, Panel C is a bar graph showing the amount of CYR61 mRNA in LRPPRC-KD and control (scramble) cells treated with vehicle or different concentrations of a TAT-hFXN fusion protein.

FIG. 7 shows acidification of the media from LRPPRC-KD and control (scramble) cells. Specifically, FIG. 7, Panel A is a photograph of a cell culture plate containing media and control (scramble) cells treated with vehicle or with different concentrations of a TAT-hFXN fusion protein. FIG. 7, Panel B is a photograph of a cell culture plate containing media and LRPPRC-KD cells treated with vehicle or with different concentrations of a TAT-hFXN fusion protein. FIG. 7, Panel C is a bar graph showing the concentration of lactate in the media of LRPPRC-KD (clone 21C) and control (scramble) cells treated with vehicle or TAT-hFXN fusion protein. FIG. 7, Panel D is a series of microscopic images showing control (scramble, SCR-5) and LRPPRC-KD (21C) cells treated with vehicle or TAT-hFXN fusion protein.

FIG. 8 is a bar graph showing the amount of CYR61 protein secreted into the media of control cells transfected with an empty vector (KD-SRBL+V), control cells transfected with hFXN (SRBL 5+hFXN), LRPPRC-KD cells transfected with an empty vector (KD-LRPPRC+V), LRPPRC-KD cells transfected with hFXN (KD-LRPPRC+V), KD-FXN cells transfected with an empty vector (KD-FXN+V) and KD-FXN cells transfected with hFXN (KD-FXN+hFXN).

FIG. 9 is a bar graph showing the amount of FXN protein per total cellular protein in the WT mouse ES clone and the homozygous mouse ES clone B9-46 which have been treated with an agent to induce the FXN knockout or a control.

FIG. 10 shows the amount of CYR61 in the ES mouse FXN knockout cells. Specifically, FIG. 10, Panel A is a bar graph showing the amount of CYR61 mRNA in ES homozygous mouse ES clone B9 before and after treatment with an agent to induce the FXN knockout. FIG. 10, Panel B is a bar graph showing the amount of CYR61 secreted into the media of WT mouse ES cells and the homozygous mouse ES B9 cells with the FXN gene knocked out.

### DETAILED DESCRIPTION

The present disclosure provides methods of treating Leigh Syndrome, French Canadian Type (LSFC), t comprising administering to a subject in need thereof an effective amount of a frataxin (FXN) therapeutic compound, such that the LSFC in the subject is treated. The present disclosure is based, at least in part, on a surprising discovery that mRNA levels of certain genes, such as ADAMTS1, ATF3, CYR61, NR4a1, EGR1, EGR2, EGR3 and MAOA, are altered in both LRPPRC- and FXN-deficient cells, indicating that expression of these genes is modulated by both LRPPRC and FXN. In particular, it was discovered that the expression of CYR61 is increased in both LRPPRC- and FXN-deficient cells, and that this increase in CYR61 expression may be reversed by treatment with a frataxin therapeutic compound (e.g., a fusion protein comprising frataxin, such as a TAT-hFXN fusion protein). The present disclosure is further based, at least in part, on a surprising discovery that LRPPRC-deficient cells are characterized by a mitochondrial impairment, as evidenced by acidification of cell growth media, and that this mitochondrial impairment may be reversed by providing to the cells a frataxin therapeutic compound (e.g., a fusion protein comprising frataxin, such as TAT-hFXN fusion protein). The above described discoveries indicate that certain molecular and cellular changes are associated with deficiencies in both LRPPRC and FXN, and that these changes may be mitigated and/or reversed by administering a frataxin supplementation therapy, e.g., a frataxin fusion protein. The above described discoveries also indicate that LSFC, which is associated with a deficiency in LRPPRC, may be treated by administering a frataxin supplementation therapy, e.g., a frataxin fusion protein.

I. Frataxin (FXN) Supplementation Therapeutics

Methods provided by the present disclosure involve frataxin supplementation therapy, and comprise administering to a subject in need thereof an effective amount of a frataxin (FXN) therapeutic compound.

As used herein, the term “frataxin supplementation therapy” or “FXN supplementation therapy” refers to supplementation of frataxin in a subject, which results in increased levels, expression and/or activity of frataxin in the subject. Frataxin supplementation therapy involves the administration of a frataxin therapeutic compound to a subject in need thereof.

The term “frataxin therapeutic compound”, which is used interchangeably herein with the terms “FXN therapeutic compound”, “FXN compound” or “frataxin compound”, refers to any polypeptide that comprises, or any nucleic acid construct that is capable of providing a frataxin (FXN) protein.

Frataxin, or FXN, e.g., human FXN (hFXN), is associated with a disorder Friedreich's Ataxia (FRDA). FRDA is a genetic, progressive neurodegenerative disorder caused by a mutation in the gene encoding frataxin. Frataxin is an essential and phylogenetically conserved protein that is found in cells throughout the body, with the highest levels in the heart, spinal cord, liver, pancreas, and skeletal muscle. Frataxin is encoded in the nucleus, expressed in the cytoplasm and imported into the mitochondria where it is processed to the mature form. In humans, the 210-amino acid full-length hFXN (hFXN₁₋₂₁₀23.1 kDa) contains a typical mitochondrial targeting sequence (MTS, SEQ ID NO: 3) at the amino terminus that is processed in a 2-step cleavage by the mitochondrial matrix processing peptidase (MPP) as it is imported into the mitochondrial matrix. The resulting protein is a 130-amino acid, 14.2 kDa mature hFXN protein (hFXN₈₁₋₂₁₀). Amino acid sequences of the full-length hFXN and mature hFXN are shown in Table 1 below.

A “frataxin protein” or “FXN protein”, as used herein, encompasses full length or mature frataxin from any subject (e.g., human frataxin, e.g., full-length or mature hFXN) or a variant, fragment or derivative of frataxin (e.g., biologically active fragment of frataxin). In some embodiments, a frataxin protein is a human frataxin or a variant, fragment or derivative thereof, e.g., a full-length hFXN (SEQ ID NO: 1) or mature hFXN (SEQ ID NO: 2), as described herein, or a variant, fragment or derivative thereof.

TABLE 1
Amino acid sequences of human frataxin

| SEQ ID NO. | Protein | Amino Acid Sequence |
|---|---|---|
| 1 | Full-length | MWTLGRRAVAGLLASPSPAQAQTLTRVPRPAELAPLCGRRGL |
| | hFXN | RTDIDATCTPRRASSNQRGLNQIWNVKKQSVYLMNLRKSGTL |
| | hFXN₁₋₂₁₀ | GHPGSLDETTYERLAEETLDSLAEFFEDLADKPYTFEDYDVSF |
| | | GSGVLTVKLGGDLGTYVINKQTPNKQIWLSSPSSGPKRYDWT |
| | | GKNWVYSHDGVSLHELLAAELTKALKTKLDLSSLAYSGKDA |
| 2 | Mature | SGTLGHPGSLDETTYERLAEETLDSLAEFFEDLADKPYTFEDY |
| | hFXN | DVSFGSGVLTVKLGGDLGTYVINKQTPNKQIWLSSPSSGPKRY |
| | hFXN₈₁₋₂₁₀ | DWTGKNWVYSHDGVSLHELLAAELTKALKTKLDLSSLAYSG |
| | | KDA |
| 3 | MTS of | MWTLGRRAVAGLLASPSPAQAQTLTRVPRPAELAPLCGRRGL |
| | hFXN | RTDIDATCTPRRASSNQRGLNQIWNVKKQSVYLMNLRK |
| | hFXN₁₋₈₀ | |

In some embodiments, a “frataxin therapeutic compound”, “FXN therapeutic compound”, or “FXN compound” of the disclosure comprises a polypeptide comprising frataxin, such as human frataxin, e.g., full-length human frataxin (SEQ ID NO: 1) or mature human frataxin (SEQ ID NO: 2), or a variant, fragment (e.g., a biologically active fragment of frataxin) or derivative thereof In some embodiments, the “frataxin therapeutic compound”, “FXN therapeutic compound” or “FXN compound” comprises a polypeptide consisting of frataxin, such as human frataxin, e.g., full-length human frataxin (SEQ ID NO: 1) or mature human frataxin (SEQ ID NO: 2), or a variant, fragment (e.g., a biologically active fragment of frataxin) or derivative thereof In some embodiments, the “frataxin therapeutic compound”, “FXN therapeutic compound” or “FXN compound” comprises a polypeptide comprising an amino acid sequence with at least about 85%, at least about 90%, at least about 95% or at least about 99% sequence identity to SEQ ID NO: 1 or SEQ ID NO: 2.

In some embodiments, the “frataxin therapeutic compound”, “FXN therapeutic compound” or “FXN compound” comprises a frataxin mutant, e.g., frataxin comprising at least one point mutation. Exemplary point mutations that may be comprised in frataxin are described, e.g., in U.S. Pat. No. 9,217,019, the entire contents of which are hereby incorporated herein by reference. In one specific embodiment, a frataxin mutant may comprise a mutation at the amino acid position 147 of SEQ ID NO: 1 or position 67 of SEQ ID NO: 2. For example, the lysine (K) residue at amino acid position 147 of SEQ ID NO: 1 or at amino acid position 67 of SEQ ID NO: 2 may be substituted with a different amino acid residue, such as a histidine, serine, threonine, asparagine, glutamine, glycine, alanine, valine, isoleucine, leucine, methionine, phenylalanine, tyrosine, tryptophan, cysteine, proline, aspartic acid, or glutamic acid residue. In one embodiment, the lysine (K) residue at amino acid position 147 of SEQ ID NO: 1 or at amino acid position 67 of SEQ ID NO: 2 may be substituted with an arginine (R) residue. The term “FXN therapeutic compound”, “frataxin therapeutic compound” or “FXN compound” also encompasses a frataxin fusion protein.

As used herein, the term “frataxin fusion protein” or “FXN fusion protein” refers to a protein comprising full length frataxin, or a variant, fragment (e.g., a biologically active fragment) or derivative of frataxin, fused to at least one different amino acid sequence, e.g., a full length protein or a fragment of a different protein, or to a peptide. In some embodiments, a frataxin fusion protein comprises full-length hFXN (SEQ ID NO: 1) or mature hFXN (SEQ ID NO: 2) as described herein, fused to at least one different amino acid sequence, e.g., a full length protein or a fragment of a different protein, or to a peptide. In some embodiments, the frataxin fusion protein comprises an amino acid sequence with at least about 85%, at least about 90%, at least about 95% or at least about 99% sequence identity to SEQ ID NO: 1 or SEQ ID NO: 2 fused to at least one different amino acid sequence, e.g., a full length protein or a fragment of a different protein, or to a peptide. In some embodiments, the frataxin fusion protein comprises frataxin, or a variant, fragment (e.g., a biologically active fragment) or derivative thereof, fused to a cell penetrating peptide (CPP) as described herein. In some embodiments, the CPP is the transduction domain of HIV-TAT (e.g., SEQ ID NO: 4).

In some examples, the at least one different amino acid sequence comprised in a fusion protein of the present disclosure may comprise a cell penetrating peptide (CPP). Cell penetrating peptides (CPPs) are short peptide sequences, typically between 5 and 30 amino acids long, that can facilitate cellular intake of various molecular cargo, such as proteins. When present in a fusion protein, a CPP may facilitate the delivery of the fusion protein to a cell, e.g., a recipient cell. A CPP useful in the context of the present invention may be any CPP known to a person skilled in the art. For example, a CPP comprised in the fusion protein of the present disclosure may be any CPP listed in the Database of Cell-Penetrating Peptides CPPsite 2.0, the entire contents of which are hereby incorporated herein by reference. In some embodiments, a CPP useful in the context of the present invention may comprise an aromatic cationic peptide as described, e.g., in U.S. Pat. No. 10,576,124, the entire contents of which are hereby incorporated herein by reference. For example, an aromatic cationic peptide may be 2′, 6′ dimethyl-Tyr-D-Arg-Phe-Lys-NH₂.

In some embodiments, a CPP useful in the context of the present invention may be selected from the group consisting of HIV-TAT (which may also be referred to herein as “TAT”), the transduction domain of HIV-TAT, galanin, mastoparan, transportan, penetratin, polyarginine, or VP22. In one embodiment, the CPP comprises the transduction domain of HIV-TAT having an amino acid sequence YGRKKRRQRRR (SEQ ID NO: 4). Table 2 below lists amino acid sequences of the exemplary CPPs.

TABLE 2
Exemplary CPPs and corresponding amino acid sequences

| SEQ ID NO. | CPP | Amino Acid Sequence |
|---|---|---|
| 4 | HIV-TAT | YGRKKRRQRRR |
| 5 | HIV-TAT + M | MYGRKKRRQRRR |
| 6 | Galanin | GWTLNSAGYLLGPHAVGNHRSFSDKNGLTS |
| 7 | Mastoparan | INLKALAALAKKIL-NH₂ |
| 8 | Transportan | GWTLNSAGYLLGKINLKALAALAKKIL |
| 9 | Penetratin | RQIKIWFQNRRMKWKKGG |
| 10 | Penetratin | RQIKIWFQNRRMKWKK |
| | (16-mer) | |
| 11 | Polyarginine | RRRRRRRRR |
| 12 | VP22 | DAATATRGRSAASRPTERPRAPARSASRPRRP |
| | | VE |
| 13 | VP22 | NAATATRGRSAASRPTQRPRAPARSASRPRRP |
| | | VQ |

In some embodiments, the CPP comprised in a fusion protein useful in the context of the present disclosure may comprise, or consist of, an amino acid sequence with at least about 85%, at least about 90%, at least about 95% or at least about 99% sequence identity to any one of SEQ ID NOs: 4-13 as listed in Table 2. In one specific embodiment, the CPP may comprise, or consist of, an amino acid sequence with at least about 85%, at least about 90%, at least about 95% or at least about 99% sequence identity to SEQ ID NO: 4. In one specific embodiment, the CPP may comprise, or consist of, an amino acid sequence with at least about 85%, at least about 90%, at least about 95% or at least about 99% sequence identity to SEQ ID NO: 5. In some embodiments, the CPP may comprise an amino acid sequence of any one of SEQ ID NOs: 4-13, e.g., SEQ ID NO: 4. In some embodiments, the CPP comprises the amino acid sequence of SEQ ID NO: 4.

In some embodiments, the fusion protein may comprise an amino acid sequence with at least about 85%, at least about 90%, at least about 95% or at least about 99% sequence identity to SEQ ID NO: 2 and at least one different amino acid sequence. The at least one different amino acid sequence may comprise a mitochondrial targeting sequence (MTS), i.e., an amino acid sequence that facilitates delivery of the fusion protein to mitochondria. An MTS may be a peptide of about 10-70 amino acids, and, in some examples, may comprise an alternative pattern of hydrophobic and positively charged amino acids that forms an amphipathic helix.

In some embodiments, a fusion protein useful in the context of the present disclosure may comprise an amino acid sequence with at least about 85%, at least about 90%, at least about 95% or at least about 99% sequence identity to SEQ ID NO: 2 and a native MTS of frataxin, i.e., SEQ ID NO: 3. In some embodiments, a fusion protein useful in the context of the present disclosure may comprise an amino acid sequence with at least about 85%, at least about 90%, at least about 95% or at least about 99% sequence identity to SEQ ID NO: 2 and an amino acid sequence with at least about 85%, at least about 90%, at least about 95% or at least about 99% sequence identity to SEQ ID NO: 3.

A fusion protein useful in the context of the present disclosure may also comprise an amino acid sequence with at least about 85%, at least about 90%, at least about 95% or at least about 99% sequence identity to SEQ ID NO: 2 and a heterologous MTS sequence, i.e., MTS that is not naturally associated with frataxin. For example, a fusion protein may comprise an amino acid sequence with at least about 85%, at least about 90%, at least about 95% or at least about 99% sequence identity to SEQ ID NO: 2 and an MTS derived from citrate synthase or lipoamide dehydrogenase (LAD), as described in, e.g., U.S. Pat. No. 8,912,147, the entire contents of which are incorporated herein by reference.

In some embodiments, a fusion protein of the present disclosure may comprise an amino acid sequence with at least about 85%, at least about 90%, at least about 95% or at least about 99% sequence identity to SEQ ID NO: 2 and an at least one different amino acid sequence, wherein the at least one different amino acid sequence comprises, or consists of, an MTS as described herein, e.g., SEQ ID NO: 3, and a CPP as described herein, e.g., SEQ ID NO: 4.

In some embodiments, the term “frataxin therapeutic compound”, “FXN therapeutic compound” or “FXN compound”, as used herein, comprises, or consists of, the TAT-hFXN fusion protein described in U.S. Provisional Patent Application No. 62/880,073, filed on Jul. 29, 2019 and U.S. Provisional Patent Application No. 62/891,029, filed on Aug. 23, 2019, the entire contents of each of which are hereby incorporated herein by reference. This fusion protein comprises the transduction domain of HIV-TAT protein (SEQ ID NO: 4) as CPP, linked through a GG linker to the N-terminus of human frataxin. The human frataxin in this TAT-hFXN fusion protein is the full-length human frataxin (hFXN₁₋₂₁₀; SEQ ID NO: 1) as described herein. As this TAT-hFXN fusion protein is imported into the mitochondrial matrix, it is cleaved at amino acid 81, yielding the mature human frataxin (hFXN81-210; SEQ ID NO: 2). This TAT-hFXN fusion protein has the amino acid sequence of SEQ ID NO: 14, as shown below:

| SEQ ID NO. | Protein | Amino Acid Sequence |
|---|---|---|
| 14 | TAT- | MYGRKKRRQRRRGGMWTLGRRAVAGLLASPSPAQAQTLTRV |
| | hFXN | PRPAELAPLCGRRGLRTDIDATCTPRRASSNQRGLNQIWNVKK |
| | fusion | QSVYLMNLRKSGTLGHPGSLDETTYERLAEETLDSLAEFFEDL |
| | protein | ADKPYTFEDYDVSFGSGVLTVKLGGDLGTYVINKQTPNKQIWL |
| | | SSPSSGPKRYDWTGKNWVYSHDGVSLHELLAAELTKALKTKL |
| | | DLSSLAYSGKDA |

In some embodiments, the term “frataxin therapeutic compound”, “FXN therapeutic compound” or “FXN compound”, as used herein, may comprise, or consist of, an amino acid sequence with at least about 85%, at least about 90%, at least about 95% or at least about 99% sequence identity to SEQ ID NO: 14, or a variant, fragment or derivative thereof.

In some embodiments, the term “frataxin therapeutic compound”, “FXN therapeutic compound” or “FXN compound”, as used herein, also encompasses a nucleic acid sequence, e.g., a vector, encoding a polypeptide that comprises, or consists of, an amino acid sequence with at least about 85%, at least about 90%, at least about 95% or at least about 99% sequence identity to SEQ ID NO: 1, SEQ ID NO: 2 or SEQ ID NO: 14, or a variant, fragment or derivative thereof. In some embodiments, the “frataxin therapeutic compound” comprises a nucleic acid sequence, e.g., a vector, encoding a polypeptide that comprises, or consists of, the amino acid sequence of SEQ ID NO: 1, SEQ ID NO: 2 or SEQ ID NO: 14. In some embodiments, the “frataxin therapeutic compound” comprises a nucleic acid sequence, e.g., a vector, encoding a polypeptide that comprises, or consists of, the amino acid sequence of SEQ ID NO: 14. In some aspects, the nucleic acid sequence may be in a cell, e.g., a cell that is capable of producing a frataxin protein, e.g., hFXN or producing a frataxin fusion protein, e.g., comprising hFXN.

In some embodiments, the “FXN compound”, “frataxin therapeutic compound” or “FXN therapeutic compound” comprises a polypeptide comprising, or consisting of, a frataxin fragment. In some embodiments, the “FXN compound”, “frataxin therapeutic compound” or “FXN therapeutic compound” comprises a nucleic acid sequence encoding a polypeptide comprising, or consisting of, a frataxin fragment. The term “frataxin fragment”, used interchangeably herein with the terms “fragment of frataxin” and “a portion of frataxin”, encompasses any polypeptide that is shorter than a full-length frataxin or mature frataxin, e.g., shorter than human full-length frataxin (SEQ ID NO: 1) or human mature frataxin (SEQ ID NO: 2).

In preferred embodiments, a frataxin fragment may have a biological activity of frataxin, i.e., a biologically active fragment of frataxin. For example, in some embodiments, a frataxin fragment may have substantially the same biological activity as full length or mature frataxin, e.g., human full-length frataxin (SEQ ID NO: 1) or human mature frataxin (SEQ ID NO: 2). In other embodiments, a frataxin fragment may have partial biological activity of frataxin, for example, about 30% to about 100%, e.g., about 30% to about 60%, about 50% to about 80% or about 60% to about 95% of the biological activity of full length or mature frataxin, e.g., human full-length frataxin (SEQ ID NO; 1) or human mature frataxin (SEQ ID NO: 2).

In accordance with methods of the present invention, the frataxin may be provided through delivery of an frataxin polypeptide or through delivery of a nucleic acid encoding an frataxin polypeptide. Frataxin may be full length frataxin, a frataxin fragment, a mature frataxin fragment, a frataxin fusion protein, or a variant, fragment or derivative thereof. A frataxin fusion protein may comprise a frataxin fragment or full length frataxin, fused to a different protein or to a fragment thereof, or to a peptide.

An example of useful peptides for protein delivery to be a component of a frataxin fusion protein as described herein are cell-penetrating peptides (CPP), protein transduction domains, amphipathic peptides such as MAP, KALA, ppTG20, proline-rich peptides, MPG-derived peptides, Pep-1, and also loligomers, arginine-rich peptides and calcitonin-derived peptides.

CPPs are short peptide sequences, generally 5-30 amino acids (aa) long, which can translocate across the cell membrane and are used as carriers for intracellular cargo delivery. A CPP comprised in a frataxin fusion protein used herein for administration to a subject affected by LSFC may be any CPP known to the person skilled in the art, such as, for example, the transduction domain of HIV-TAT (also referred to herein as TAT), galanin, mastoparan, transportan, penetratin, polyarginine, or VP22.

In some embodiments, a frataxin fusion protein may comprise frataxin, or a variant, fragment or derivative thereof, and CPP, wherein the CPP is fused to the frataxin at the N-terminus of the frataxin. In other embodiments, a frataxin fusion protein may comprise frataxin, or a variant, fragment or derivative thereof, and CPP, wherein the CPP is fused to frataxin at the C-terminus of the frataxin. The CPP may be fused to the frataxin directly, or indirectly, e.g., through a linker). Exemplary linkers that may be used to connect frataxin and CPP in a frataxin fusion protein are described herein and shown in Table 3 below.

In a TAT-FXN fusion protein, TAT or a fragment of TAT may be directly or indirectly (through a linker) linked to either the N- or the C-terminus of frataxin. One example of a TAT-FXN fusion protein is the frataxin fusion protein described in the Examples.

As used herein, the term “directly” means that the (first or last) amino acid at the terminal end (N or C-terminal end) of the polypeptide is fused to the (first or last) amino acid at the terminal end (N or C-terminal end) of the heterologous polypeptide. In other words, in this embodiment, the last amino acid of the C-terminal end of said polypeptide is directly linked by a covalent bond to the first amino acid of the N-terminal end of said heterologous polypeptide, or the first amino acid of the N-terminal end of said polypeptide is directly linked by a covalent bond to the last amino acid of the C-terminal end of said heterologous polypeptide.

As used herein, the term “linker”, which may be used interchangeably with the term “spacer”, refers to a sequence of at least one amino acid that links the polypeptides and proteins comprised in the frataxin fusion protein utilized in the present disclosure. Such a linker may be useful to prevent steric hindrances. The length of a linker may vary from 2 to 31 amino acids, optimized for each condition so that the linker does not impose any constraints on the conformation or interactions of the linked partners of the fusion protein. Linkers have been described in the literature both as endogenous, naturally occurring, playing a role in separating domains within a protein or for the formation of dimers. Alternatively, linkers have been described in recombinant technology for the generation of fusion proteins. Non-limiting examples of linkers that may be be comprised in the fusion protein of the present disclosure, e.g., may connect the CPP, frataxin and/or MTS, are shown in Table 3 below.

TABLE 3
Linkers

| SEQ ID NO. | Linker Amino Acid Sequence |
|---|---|
| 15 | GGGGSLVPRGSGGGGS |
| 16 | GSGSGS |
| 17 | GSGSGSGS |
| 18 | GGSGGHMGSGG |
| 19 | GGSGGSGGSGG |
| 20 | GGSGG |
| 21 | GGGSEGGGSEGGGSEGGG |
| 22 | AAGAATAA |
| 23 | GGGGG |
| 24 | GGSSG |
| 25 | GSGGGTGGGSG |
| | GT |
| | GG |

By way of example, the transduction domain of HIV-TAT (HIV-TAT peptide) may be fused to frataxin through a di-peptide, Gly-Gly (GG) linker.

The variants referred to in this disclosure optionally include conservatively substituted variants that apply to both amino acid and nucleic acid sequences. With respect to nucleic acid sequences, conservatively modified variants refer to those nucleic acids which encode identical or essentially identical amino acid sequences. Specifically, degenerate codon substitutions may be achieved by generating sequences in which the third position of one or more selected (or all) codons is substituted with mixed base and/or deoxyinosine residues. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are silent variations, which are one species of conservatively modified variations. Every nucleic acid sequence herein which encodes a polypeptide also describes every possible silent variation of the nucleic acid. One of skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With regard to conservative substitution of amino acid sequences, one of skill in the art will recognize that individual substitutions, deletions or additions to a nucleic acid, peptide, polypeptide, or protein sequence which alters, adds or deletes a single amino acid or a small percentage of amino acids in the encoded sequence is a conservatively modified variant where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following groups each contain amino acids that are conservative substitutions for one another:
1) Alanine (A), Glycine (G);
2) Serine (S), Threonine (T);
3) Aspartic acid (D), Glutamic acid (E);
4) Asparagine (N), Glutamine (Q);
5) Cysteine (C), Methionine (M);
6) Arginine (R), Lysine (K), Histidine (H);
7) Isoleucine (1), Leucine (L), Valine (V); and
8) Phenylalanine (F), Tyrosine (Y), Tryptophan (W).

The term “derivative”, as used herein, refers to amino acid sequences (polypeptides), which differ from the polypeptides specifically defined in the present disclosure by insertions, deletions, substitutions and modifications of amino acid residues that do not alter the activity of the original polypeptides. It should be appreciated that by the terms “insertion/s”, “deletion/s” or “substitution/s”, as used herein it is meant any addition, deletion or replacement, respectively, of amino acid residues to the polypeptides of between 1 to 50 amino acid residues, or between 1 to 10 amino acid residues. More particularly, insertion/s, deletion/s or substitution/s may be of any one of 1, 2, 3, 4, 5, 6, 7, 8, 9, or 10 amino acid residues. It should be noted that the insertion/s, deletion/s or substitution/s may occur in any position of the modified peptide, as well as in any of the N′ or C′ termini thereof.

The term “derivative” also refers to homologues, variants and analogues thereof, as well as covalent modifications of a polypeptide. A derivative, a variant and an analogue of any one of the proteins or the peptides comprised in the fusion protein provided in the present disclosure, e.g., frataxin or a frataxin fragment described herein, will preferably have the same or substantially the same biological activity as its native form.
II. Compositions and Modes of Administration

In accordance with methods of the present invention, a frataxin therapeutic compound may be administered as a part of a pharmaceutical composition comprising a frataxin therapeutic compound and a pharmaceutically acceptable diluent, carrier, additive and/or excipient.

Preparation of pharmaceutical compositions is discussed in, for example, in Hoover, John E.(eds.) Remington's Pharmaceutical Sciences, Mack Publishing Co., Easton, Pa. 18^{th} edition (1990), and in Liberman, H. A. and Lachman, L. (eds.) Pharmaceutical Dosage Forms, Marcel Decker, New York, N.Y. (1989), the entire contents of each of which are hereby incorporated herein by reference.

A pharmaceutical composition may be delivered to cells in vitro, for example by contacting the cells with the pharmaceutical composition. Alternatively, for delivery in vivo, the pharmaceutical composition in accordance with methods of the present disclosure may be administered parenterally in dosage unit formulations containing conventional nontoxic pharmaceutically acceptable carriers, adjuvants, and vehicles as desired. The term parenteral as used herein includes subcutaneous, intravenous, intramuscular, or intrasternal injection, or infusion techniques.

Injectable pharmaceutical compositions, for example, sterile injectable aqueous or oleaginous suspensions, may be formulated according to the techniques known in the art using suitable dispersing or wetting agents and suspending agents. The sterile injectable pharmaceutical composition may also be a sterile injectable solution or a suspension in a nontoxic parenterally acceptable diluent or solvent, for example, 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids, such as oleic acid, are useful in the preparation of injectables. Dimethyl acetamide, surfactants including ionic and non-ionic detergents, and polyethylene glycols can be used. Mixtures of solvents and wetting agents such as those discussed above are also useful.

Pharmaceutical compositions for parenteral administration may be in the form of aqueous or non-aqueous isotonic sterile injection solutions or suspensions. These solutions and suspensions may be prepared from sterile powders or granules having one or more of the carriers or diluents. A frataxin therapeutic compound to be administered in accordance with methods of the present invention may be formulated in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

A frataxin therapeutic compound may also be administered in accordance with methods of the present invention through an expression vector for producing a frataxin therapeutic compound in a cell (such as a mammalian, bacterial or fungal cell). The expression vector may comprise a nucleic acid encoding a frataxin therapeutic compound, e.g., a frataxin protein or frataxin fusion protein comprising a frataxin protein, as described herein. By way of example, the expression vector may be a retroviral vector, a DNA vector, a plasmid, an RNA vector, an adenoviral vector, an adenovirus associated vector, a lentiviral vector, a phagemid, a baculovirus, or any combination thereof.

As used herein, “nucleic acids” is interchangeable with the term “polynucleotide(s)” and it generally refers to any polyribonucleotide or poly-deoxyribonucleotide, which may be unmodified RNA or DNA or modified RNA or DNA or any combination thereof “Nucleic acids” include, without limitation, single- and double-stranded nucleic acids.

Vectors may enable the integration of DNA fragments or nucleic acid sequences into the genome of the host or enable expression of genetic elements that are not integrated. Vectors are typically self-replicating DNA or RNA constructs containing the desired nucleic acid sequences, and operably linked genetic control elements that are recognized in a suitable host cell and effect the translation of the desired spacers. Generally, the genetic control elements can include a prokaryotic promoter system or a eukaryotic promoter expression control system. Such system typically includes a transcriptional promoter and transcriptional enhancers to elevate the level of RNA expression. Vectors usually contain an origin of replication that allows the vector to replicate independently of the host cell. Accordingly, the term “control and regulatory elements” may include promoters, terminators and other expression control elements. Such regulatory elements are described in the art and known to the skilled artisan. For instance, any of a wide variety of expression control sequences that control the expression of a DNA sequence when operatively linked to it may be used in these vectors to express DNA sequences encoding any desired fusion protein as described herein. The phrase “operatively-linked” is intended to mean attached in a manner which allows for transgene transcription. The term “encoding” is intended to mean that the subject nucleic acid may be transcribed and translated into either the desired polypeptide or the subject protein in an appropriate expression system, e.g., when the subject nucleic acid is linked to appropriate control sequences such as promoter and enhancer elements in a suitable vector (e.g., an expression vector) and when the vector is introduced into an appropriate system or cell.

A vector may additionally include appropriate restriction sites, antibiotic resistance or other markers for selection of vector-containing cells. Plasmids are the most commonly used form of vector but other forms of vectors which serve an equivalent function, and which are, or become, known in the art are suitable for use herein.

A vector may also be referred to as a construct, or as recombinant nucleic acid. As referred to herein, the term “recombinant DNA”, “recombinant nucleic acid sequence” or “recombinant gene” refers to a nucleic acid comprising an open reading frame (ORF) encoding a frataxin fusion protein as described herein.

A frataxin therapeutic compound, e.g., frataxin protein or frataxin fusion protein, may also be delivered in accordance with methods of the present disclosure by viral gene replacement, which may utilize retroviral, lentiviral, or adeno-associated viral vectors, as well as adenoviruses to express the frataxin protein or frataxin fusion protein.

A frataxin therapeutic compound comprising a nucleic acid encoding a frataxin protein or frataxin fusion protein, may also be delivered in accordance with methods of the present disclosure using a non-viral delivery system. Non-limiting examples of non-viral delivery systems include, e.g., lipid-based DNA vectors, and polymeric DNA vectors, as described in Yin et al., Nature Reviews Genetics 2014, 15, 541-555, the entire contents of which are incorporated herein by reference.

It is to be understood that a “frataxin therapeutic compound” of the disclosure, e.g., a polypeptide comprising a frataxin protein or frataxin fusion protein or a polynucleotide encoding the frataxin protein or frataxin fusion protein, encompasses frataxin orthologs or homologues having a sequence homology or identity to frataxin, e.g., SEQ ID NO: 1 or SEQ ID NO: 2, of at least 50%, at least 60%, at least 65%, at least 70%, at least 75%, at least 80%, at least about 85%, at least about 90%, 91%, 92%, 93%, 94%, 95%, 96%, 97%, 98%, 99%, or higher, specifically as compared to the entire sequence of the frataxin as denoted by SEQ ID NO. 1 or SEQ ID NO: 2. Specifically, homologs that comprise or consist of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 1 or SEQ ID NO: 2, may be comprised in the frataxin therapeutic compounds described herein.

In an embodiment, a frataxin therapeutic compound, e.g., frataxin polypeptide sequence, or a fusion protein comprising frataxin polypeptide sequence, may be prepared as part of a protein delivery system. A broad overview of protein delivery systems may be found, for example, in Banga, A. K. (2015) Therapeutic Peptides and Proteins: Formulation, Processing, and Delivery Systems, 3rd ed., CRC Press, Taylor & Francis Group. Particulate systems include microspheres, microparticles, microcapsules, nanocapsules, nanospheres, and nanoparticles. Microcapsules may contain the frataxin therapeutic compound, such as frataxin, as a central core. In microspheres, the frataxin therapeutic compound may be dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm diameter and are administered subcutaneously or intramuscularly. See, for example, Kreuter, J. (1994) Colloidal Drug Delivery Systems, CRC Press, Taylor & Francis Group, New York, N.Y., pp. 219-342 (1994); and Tice & Tabibi (1992) Treatise on Controlled Drug Delivery, Chapter 7, A. Kydonieus, ed., CRC Press, Taylor & Francis Group, New York, N.Y., pp. 315-339.

It is further encompassed by the disclosure that, alternatively, in some embodiments, treatment of a subject affected by LSFC may also be achieved by upregulation of the endogenous frataxin gene, resulting in increased levels of endogenous frataxin in the cell of the subject affected by LSFC. Upregulation of an frataxin gene may be triggered through a compound, a drug, or any agent that affects the mitochondrial pathway.
III. Methods of Treating LSFC

The present disclosure provides methods for treating LSFC that comprise administering to a subject in need thereof an effective amount of a frataxin therapeutic compound, e.g., a frataxin fusion protein, or a variant, fragment, or derivative thereof, such that the LSFC is treated.

The term “treating LSFC”, as used herein, encompasses ameliorating, improving or achieving a reduction in the severity of LSFC, e.g., ameliorating, improving or achieving a reduction in at least one symptom or indicator associated with LSFC in a subject. “Treating LSFC”, as used herein, also encompasses delaying progression of LSFC, e.g., delaying appearance of at least one symptom or indicator associated with LSFC, or preventing an increase in the severity of at least one symptom or indicator associated with LSFC in a subject. In some embodiments, the at least one symptom or indicator associated with LSFC may be selected from the group consisting of developmental delay, ataxia, hypotonia, brain lesions, coma, abnormal breathing patterns, seizures, stroke-like episodes and lactic acidosis.

In an embodiment, a frataxin fusion protein which may be used in the treatment of LSFC is a TAT-hFXN fusion protein.

In one embodiment, “treating LSFC” encompasses ameliorating, improving or achieving a reduction in the severity of lactic acidosis in a subject.

The term “effective amount” of a frataxin therapeutic compound, e.g., a TAT-hFXN fusion protein (e.g., SEQ ID NO:14), is that amount which is necessary or sufficient to treat LSFC, e.g., ameliorate, improve or achieve a reduction in the severity of at least one symptom or indicator associated with LSFC. For example, an “effective amount” of a frataxin therapeutic compound is that amount which is necessary or sufficient to ameliorate, improve or achieve a reduction in the severity of lactic acidosis.

In some embodiments, the term “effective amount” of a frataxin therapeutic compound, e.g., a TAT-hFXN fusion protein (e.g., SEQ ID NO:14), may also be that amount which is necessary to modulate one or more proteins selected from the group consisting of ADAMTS1, ATF3, CYR61, NR4a1, EGR1, EGR2, EGR3, MAOA and IFN1 in a subject afflicted with LSFC. One of ordinary skill in the art can determine an effective amount of a frataxin therapeutic compound for administration to a subject. Drug dosages and regimens for treating various conditions are well known in the art. Note in this regard, for example, Goodman & Gilman's The Pharmacological Basis of Therapeutics, 1996, Ninth Edition, McGraw-Hill, New York. The effective amount of frataxin therapeutic compound may be combined with carrier materials to produce a single dosage form will vary depending upon the patient and the particular mode of administration.

In some embodiments, administration of a frataxin therapeutic compound to a subject may modulate LRPPRC in the subject. In some embodiments, contacting a cell in vitro, ex vivo, or in vivo with a frataxin therapeutic compound may modulate LRPPRC in the cell, e.g., as described in the Examples. In some embodiments, administration of a frataxin therapeutic compound to a subject may modulate SLIRP in the subject. In some embodiments, contacting a cell in vitro, ex vivo, or in vivo with a frataxin therapeutic compound may modulate SLIRP in the cell, e.g., as described in the Examples.

The term “modulate LRPPRC”, as used herein, refers to causing the amount of LRPPRC (e.g., the expression level) or the level of the protein activity to change (e.g., increase or decrease) following administration of a frataxin therapeutic compound, e.g., a TAT-hFXN fusion protein (e.g., SEQ ID NO:14), to a subject, or in a cell being contacted with a frataxin therapeutic compound.

The term “modulate SLIRP”, as used herein, refers to causing the amount of SLIRP (e.g., the expression level) or the level of the protein activity to change (e.g., increase or decrease) following administration of a frataxin therapeutic compound, e.g., a TAT-hFXN fusion protein (e.g., SEQ ID NO:14), to a subject, or in a cell being contacted with a frataxin therapeutic compound.

The Examples herein provide evidence that a group of overlapping genes, referred to herein as the FXN/LRPPRC targets, have the level of their transcripts altered in the absence of either FXN or LRPPRC. Interestingly, a sub-group within this group, namely EGR1, EGR3, NR4a1, and IFN1, are all involved in the development of dorsal root ganglia and myelination.

Thus, in some embodiments of the disclosure, the frataxin therapeutic compound may modulate any one, or one or more, of the FXN/LRPPRC targets ADAMTS1, ATF3, CYR61, NR4a1, EGR1, EGR2, EGR3, MAOA and IFN1, in a subject following administration to the subject, or in a cell contacted or treated with the frataxin therapeutic compound in vivo or in vitro.

The term “modulated level” of a protein refers to a changed, e.g., increased or decreased amount or activity of a particular protein, for example LRPPRC, SLIRP, or cytochrome c oxidase (COX) family member, such as COX2, as compared to the amount or activity of the protein in a control sample, when detected by any of the techniques known to the person skilled in the art.

The “amount” of a protein, or a protein level in a subject or in a cell is “significantly” higher or lower than the normal amount of said protein, if the amount of the protein is greater or less, respectively, than the normal level (e.g., level in the healthy subject or a threshold level) by an amount greater than the standard error of the assay employed to assess amount, and preferably at least twice, and more preferably three, four, five, ten or more times that amount. Alternatively, the amount of the protein in a subject or in a cell can be considered “significantly” higher or lower than the normal amount if the amount is at least about two, and preferably at least about three, four, or five times, higher or lower, respectively, than the normal amount of the protein. In some embodiments, the amount of the protein in the subject or in a cell can be considered “significantly” higher or lower than the normal amount (e.g., amount in a healthy subject or a threshold amount) if the amount is 10%, 15%, 20%, 25%, 30%, 35%, 40%, 45%, 50% or more, higher or lower, respectively, than the normal amount of the protein. The term “modulated level” of a protein may also refer to an increased or decreased level of activity of a protein, e.g., LRPPRC protein or SLIRP protein, which may, or may not be caused by increased or decreased amounts of the protein.

The proteins which have their levels modulated by following administration of a frataxin therapeutic compound may be detected and quantitated by any suitable method for detecting proteins or polypeptides known to the person skilled in the art. Generally, proteins may be detected by a variety of immunodetection methods, such as enzyme linked immunosorbent assay (ELISA), radioimmunoassay (MA), immunoradiometric assay, fluoroimmunoassay, chemiluminescent assay, bioluminescent assay, and Western blot, though several others are well known to those of ordinary skill in the art. The steps of various useful immunodetection methods have been described in the scientific literature.

Alternatively or additionally, the proteins which have their levels modulated by a frataxin therapeutic compound compound may be detected and quantitated using protein mass spectrometry methods and instrumentation. Protein mass spectrometry (MS), which may be achieved for example by electrospray ionization (ESI) or matrix-assisted laser desorption/ionization (MALDI). Whole protein mass analysis of the proteins can be conducted using time-of-flight (TOF) MS, or Fourier transform ion cyclotron resonance (FT-ICR).

The proteins can also be measured in complex mixtures of proteins and molecules that co-exist in a biological medium or sample, however fractionation of the sample may be required and is contemplated herein. Two methods are widely used to fractionate proteins, or their peptide products from an enzymatic digestion, two-dimensional gel electrophoresis (2D Gel) and high-performance liquid chromatography (LC or HPLC). Any other suitable methods known in the art for fractionating protein mixtures are also contemplated herein.

As used herein, “changed, modulated, altered, increased or decreased” is understood as having a level of the one or more FXN/LRPPRC targets to be detected at a level that is statistically different, e.g., increased or decreased, as compared to a control sample or threshold value, e.g., from a LRPPRC healthy subject (i.e., a subject with a normal LRPPRC level), or a sample from an LRPPRC deficient subject (i.e., a subject lacking or having reduced LRPPRC expression). Changed, modulated, altered, increased or decreased, as compared to a control or threshold value, can also include a difference in the rate of change of the level of one or more FXN/LRPPRC targets obtained in a series of at least two subject samples obtained over time. Determination of statistical significance is within the ability of those skilled in the art and can include any acceptable means for determining and/or measuring statistical significance, such as, for example, the number of standard deviations from the mean that constitute a positive or negative result, an increase in the detected level of an FXN/LRPPRC target in a sample versus a control, wherein the increase is above some threshold value, or a decrease in the detected level of an FXN/LRPPRC target in a sample versus a control, wherein the decrease is below some threshold value. In some embodiments, altered expression is modulated or altered gene expression, which in the method exemplified herein presents itself as differential gene expression, also known as differential mRNA expression. Altered or modulated expression may comprise increased expression, also referred to as overexpression or upregulation, or decreased or inhibited expression, also referred to as downregulation.

In an embodiment, mtRNA is stabilized in cells ex vivo or in vivo upon administration of a frataxin therapeutic compound. Stabilization of mtRNA in a cell may be detected through for example, detection of mtRNA amounts in a cell, or analyzing the length of polyadenylated (polyA) tails.

Detection of mRNA may be performed through a variety of techniques known to the person skilled in the art, such as for example Northern blot, nuclease protection assays, in situ hybridization, and reverse transcription-polymerase chain reaction.

By way of example, analysis of polyA tail length may be performed by any nucleotide sequencing techniques known to the person skilled in the art.

In accordance with methods of the present disclosure, in some embodiments a frataxin therapeutic compound is administered to a subject, e.g., subject with LSFC, in an amount effective to treat the LSFC in the subject. In some embodiments, a frataxin therapeutic compound is administered to a subject, e.g., subject with LSFC, in an amount effective to modulate LRPPRC in the subject, or an amount effective to modulate SLIRP in the subject. One of ordinary skill in the art can determine an effective amount of frataxin therapeutic compound for administration to a subject. Drug dosages and regimens for treating various conditions are well known in the art. Note in this regard, for example, Goodman & Gilman's The Pharmacological Basis of Therapeutics, 1996, Ninth Edition, McGraw-Hill, New York. The amount of active ingredient of a frataxin therapeutic compound that can be combined with the carrier materials to produce a single dosage form will vary depending upon the patient and the particular mode of administration.

The frataxin therapeutic compound may be administered in accordance with methods of the present disclosure by a variety of methods, including, for example, orally, enterally, mucosally, percutaneously, or parenterally. Parenteral administration is preferred, especially by intravenous, intramuscular, subcutaneous, intracutaneous, intraarticular, intrathecal, and intraperitoneal infusion or injection, including continuous infusions or intermittent infusions with pumps available to those skilled in the art. Alternatively, the frataxin therapeutic compound, e.g., a fusion protein comprising frataxin polypeptide, may be administered by means of micro-encapsulated preparations, for example based on liposomes.

In some embodiments, the present disclosure also provides CYR61 as a marker whose level is correlated to frataxin (FXN) level in a cell, e.g., an LRPPRC deficient cell. CYR61 is one of the downstream targets of both FXN and LRPPRC, and the levels of CYR61 are contrary regulated by FXN or LRPPRC gene ablation followed by frataxin supplementation therapy. Specifically, as shown in Examples 7-9 and 11, the levels of CYR61 are increased by FXN or LRPPRC gene ablation and are decreased in cells deficient in FXN or LRPPRC following administration of frataxin therapeutic compound. Thus, CYR61 is both associated with LRPPRC deficiency in a subject and conversely associated with frataxin supplementation. Therefore, CYR61 can be used to determine and/or monitor the effectiveness of frataxin supplementation therapy in treating LSFC, e.g., the effectiveness of treating LSFC in a subject being administered a frataxin therapeutic compound in accordance with the methods of the present disclosure.

Accordingly, in some embodiments, the effectiveness of frataxin supplementation therapy in a subject with LSFC may be determined, evaluated, and/or monitored based on the analysis of levels of CYR61 before and after administration of a frataxin therapeutic compound to the subject. Based on the results of the analysis of CYR61 level, adjustments may be made to the frataxin supplementation therapy in a subject with LSFC to, e.g., initiate, increase, decrease or cease the frataxin supplementation therapy in the subject.

In some aspects, the present disclosure provides a method for evaluating effectiveness of frataxin (FXN) supplementation therapy in a subject with LSFC that comprises: (a) determining level of CYR61 in a sample from said subject following treatment with frataxin supplementation therapy; (b) comparing the level of CYR61 in (a) with a baseline CYR61 level; and (c) using the comparison to determine effectiveness of the frataxin supplementation therapy in said subject. The method may further comprise determining a baseline CYR61 level in a sample from a subject with LSFC obtained prior to the frataxin supplementation therapy.

As used herein, “subject” or “patient” can mean either a human or non-human animal, preferably a mammal. A human subject may be referred to as a patient.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is considered to be the inclusive “or” (having the meaning of and/or) rather than the exclusive or, and indicates at least one of, or any combination of items it conjoins.

### EXAMPLES

Procedures of molecular biology referred to herein are common methods used in the art, and can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York), the entire contents of each of which are expressly incorporated herein.

### Example 1

### Generation of an in vitro Cell Model for Leigh Syndrome French Canadian (LSFC) Type

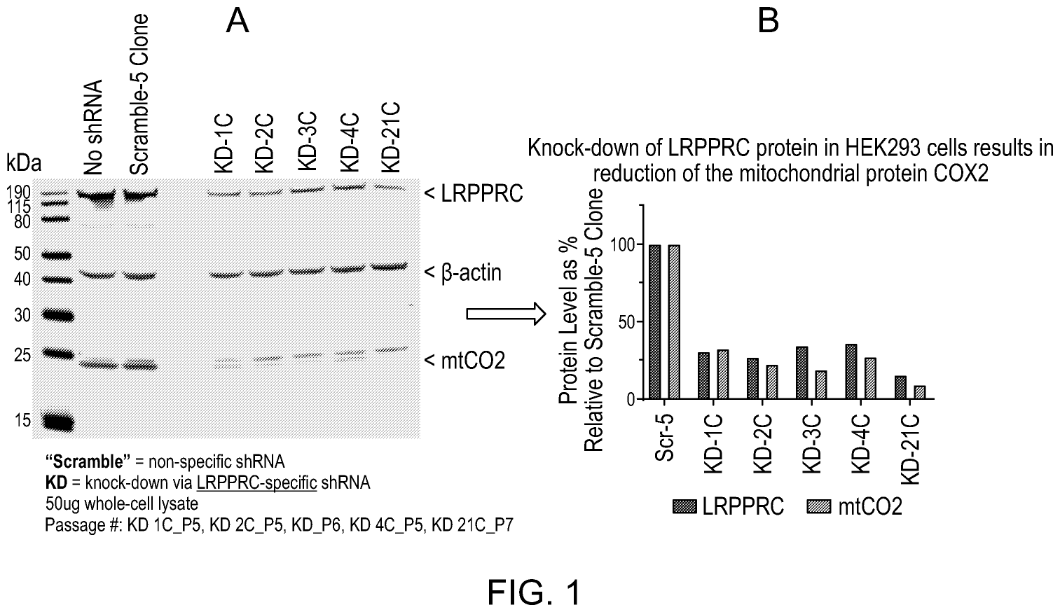

HEK293 cells were transfected with LRPPRC short hairpin (sh)RNA contained in lentiviral particles, resulting in targeted gene silencing of LRPPRC. A control cell line transfected with a scramble sequence (Scr-5) was generated in parallel. FIGS. 1A and 1B show the analysis of the cell lines generated, the control Scramble 5-Clone, and LRPPRC knock down (KD) clones KD-1C, KD-2C, KD-3C, KD-4C and KD-21C.

LRPPRC protein levels in clones KD-1C, KD-2C, KD-3C, KD-4C and KD-21C are reduced by between 70-80% when compared to the levels of LRPPRC protein in the Scramble-5 clone (see the Western blot in FIG. 1A and the graph in FIG. 1B, quantifying the protein levels).

The levels of cytochrome c oxidase subunit 2 (mtCOX2, marked as mtCO2 in the figure) were also detected in the Western blot (FIG. 1A) and quantified (FIG. 1B) and were shown to be reduced by about between 70-80% in the KD clones. Altered mtCOX2 levels and function are one of the hallmarks of Leigh Syndrome.

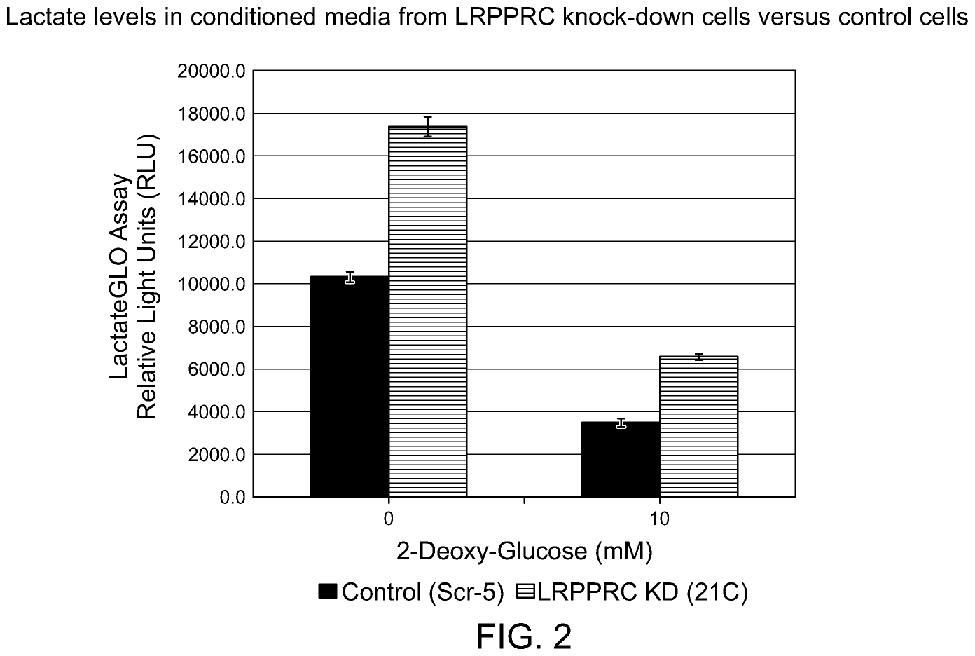

A lactate GLO assay was performed in the LRPPRC KD clone KD-21C and compared to control. HEK293 Control Cells (Scr-5) and LRPPRC KD cells (Clone 21C) were seeded at 15,000 cells per well of a 96-well plate. Cells were allowed to adhere to the plate for at least 8 hours before being treated with 2-deoxy glucose (2-DG). Forty-eight hours after treatment, 5 μL of medium was removed from each well, diluted 400-fold in PBS (phosphate buffered saline) and assayed using a commercial kit for measuring lactate content (LactateGLO assay kit, Promega) according to the manufacturer's protocol. FIG. 2 shows a plot of the lactate content (plotted as Relative Light Units) versus 2-DG concentration, in the absence of 2-DG (“0”) and in the presence of 10 mM of 2-DG (“10”). Error bars represent standard deviation between duplicate wells.

The increased levels of lactate in the KD clone demonstrate that this clone is a good phenocopy of the situation in vivo, in individuals deficient in LRPPRC.

### Example 2

### Generation of an in vitro Cell Model for Frataxin (FXN) Knock Down (KD)

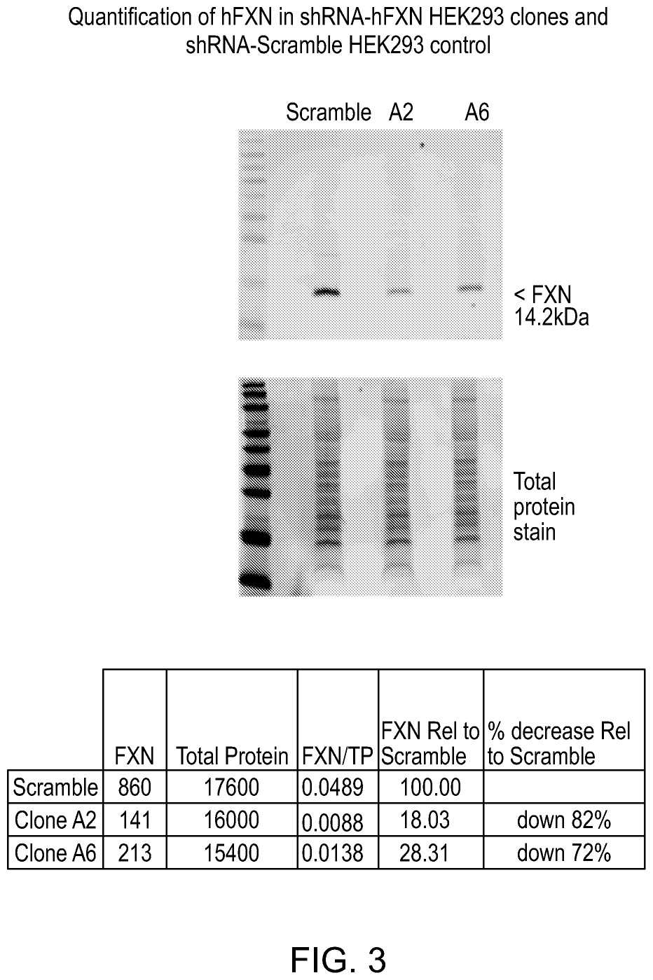

HEK-293 cells were transfected with scramble or KD-FXN constructs (clones A2 and A6) using shRNA technology, in order to repress Frataxin mRNA and protein expression in the cells, and the results are shown in FIG. 3. Expression of FXN protein in the KD-FXN clones A2 and A6 (middle and right-most lanes in the Western blot, respectively, in FIG. 3) was significantly reduced when compared to Scramble control. The table shows the quantitation of protein expression obtained from the Western blot, in FXN KD relative to control Scramble. The amount of FTX protein in Scramble control was considered 100%. A reduction of 82% and 72% FXN protein in Clones A2 and A6, respectively was observed.

### Example 3

### Overlap in Frataxin and LRPPRC Dowstream Targets

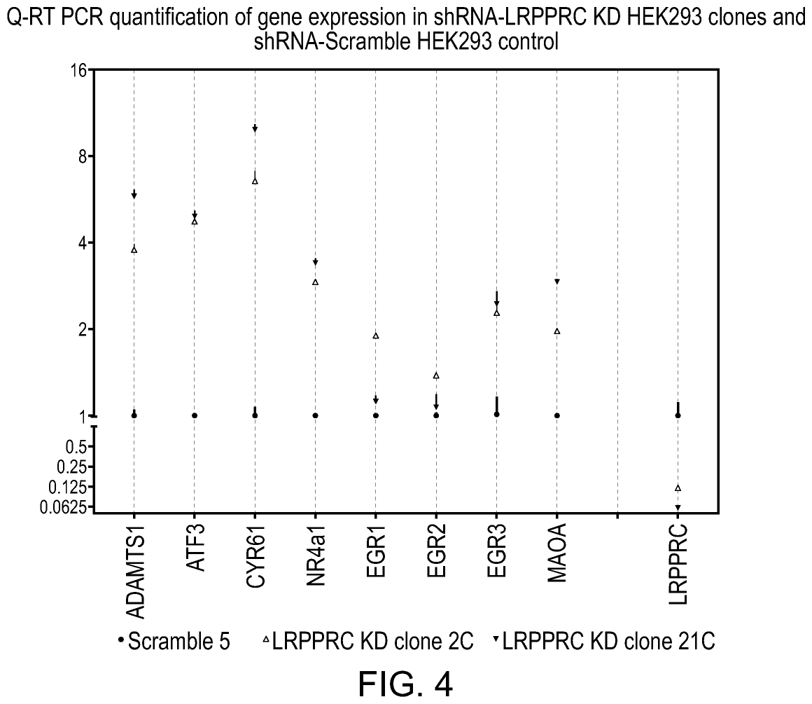

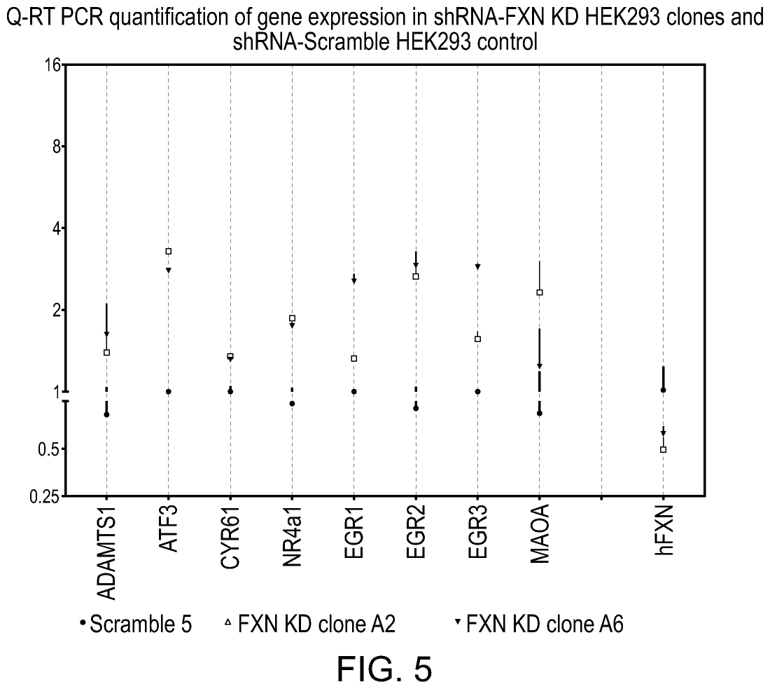

Analysis of the expression of a group of transcripts was performed in both KD-FXN (FIG. 4) and in KD-LRPPRC (FIG. 5) clones, and compared with Scramble control, respectively. The results show that mRNA expression of ADAMTS1, ATF3, CYR61, NR4a1, EGR1, EGR2, EGR3, and MAOA, also referred to herein as the FXN/LRPPRC targets, was altered in the absence of FXN as well as in the absence of LRPPRC. mRNA expression of IFN1 was also altered in both KD clones (data not shown). Therefore, expression of these transcripts is modulated by both FXN and LRPPRC. RNA techniques employed for these experiments are described below.

### RNA Extraction

Upon reaching confluency, cells were rinsed with PBS buffer. Total RNA extraction was performed using RNeasy Mini Kit (Qiagen catalog number 74104) including the optional genomic DNA removal step, according to the protocol provided by the manufacturer. Total RNA concentration was measured in solution using a Beckman Coulter DU730 UV/Vis Spectrophotometer.

### Reverse Transcription (RT)-cDNA Synthesis

Reverse transcription was performed using 4 μg of total RNA in 30 μL reaction using the Superscript IV VILO Master Mix with ezDNase Kit (Invitrogen catalogue number 766500), according to the protocol provided by the manufacturer.

### Quantitative Real-time Polymerase Chain Reaction (PCR)

Quantitative PCR, or real-time (RT) PCR, used herein interchangeably, was performed using the Quant Studio 5 automated system (Applied Biosystems). The reaction master mix was TaqMan Fast Advanced Master mix (ThermoFisher 4444557) and the plates were MicroAmp Fast 96-Well Reaction Plate (ThermoFisher 4346907). In general, each reaction (each well) consisted of: 10 μL Master Mix(20×)+0.33 μL Housekeeping Gene Primer/Probe(60×)+1 μL Target gene Primer/Probe(20×)+6.67 μL Nuclease Free H₂O+2 μL cDNA (approximately 25 ng). The PCR cycle included a 2-minute UNG (from the uracil-DNA glycosylases family, used for removal of uracil) incubation at 50° C., a 2-minute incubation at 95° C. for polymerase activation, and 40 PCR cycles of 1 second at 95° C. and 20 seconds at 60° C.

The PCR reaction used forward and reverse primers. By way of example, the forward primers are between 18 and 22 nucleotides long, and may comprise 15, 16, 17, 18, 19, 20, or 21 nucleotides identical to the target nucleic acid. The reverse primer may be complementary to the target nucleic acid. The reverse primer may also comprise a sequence complementary to an adaptor sequence.

### Example 4

### Treatment of LRPPRC Knock Down (KD) Clones with a TAT-hFXN Fusion Protein

A TAT-hFXN fusion protein was developed as a Frataxin protein supplementation therapy for the treatment of the mitochondrial disease Friedreich's Ataxia (FRDA). The TAT-hFXN fusion protein has been shown to rescue disease phenotypes associated with FRDA in cells and animals, and its localization in the cell was originally described as mitochondrial, as described in U.S. Pat. No. 8,283,444 and Vyas et al. (2012) Hum. Mol. Genet. 21, 1230-1247, the entire contents of which are expressly incorporated herein by reference.

Full-length human frataxin protein (hFXN₁₋₂₁₀) is encoded in the nucleus and is synthesized as a 210-amino acid (aa) precursor protein, having the sequence provided in SEQ ID NO:1. Following its expression, full-length hFXN₁₋₂₁₀ is directed to the mitochondria by its N-terminal 80 aa mitochondrial targeting sequence (MTS), where it is actively imported and proteolytically processed to yield the 130 aa mature form, or active fragment, of the protein (hFXN₁₋₂₁₀), with a predicted molecular weight of 14.2 kDa and a sequence as provided in SEQ ID NO.2. The FXN mature form ultimately resides in the mitochondrial matrix. TAT-hFXN fusion protein also undergoes intra-mitochondrial proteolytic processing, and the FXN 130 aa mature form is found in the mitochondria of cells treated with the TAT-hFXN fusion protein.

TABLE 4
Sequences of Frataxin and the Transduction Domain of HIV-TAT

| SEQ ID | | |
|---|---|---|
| NO. | Name | Amino Acid Sequence |
| 1 | Full-length | MWTLGRRAVAGLLASPSPAQAQTLTRVPRPAELAPLCGRR |
| | hFXN | GLRTDIDATCTPRRASSNQRGLNQIWNVKKQSVYLMNLRK |
| | (hFXN₁₋₂₁₀) | SGTLGHPGSLDETTYERLAEETLDSLAEFFEDLADKPYTFE |
| | | DYDVSFGSGVLTVKLGGDLGTYVINKQTPNKQIWLSSPSSG |
| | | PKRYDWTGKNWVYSHDGVSLHELLAAELTKALKTKLDLS |
| | | SLAYSGKDA |
| 2 | Mature hFXN | SGTLGHPGSLDETTYERLAEETLDSLAEFFEDLADKPYTFE |
| | (hFXN₈₁₋₂₁₀) | DYDVSFGSGVLTVKLGGDLGTYVINKQTPNKQIWLSSPSSG |
| | | PKRYDWTGKNWVYSHDGVSLHELLAAELTKALKTKLDLS |
| | | SLAYSGKDA |
| 4 | HIV-TAT | YGRKKRRQRRR |

The LRPPRC KD clones generated in Example 1 are grown to 100% confluence and treated with 500 nM TAT-hFXN fusion protein or an equal volume of vehicle (50 mM Sodium Acetate, pH5, 1% Propylene Glycol) delivered in 2.5 mL of FBS/puromycin-free DMEM medium. After 2.5 hours, medium with TAT-hFXN fusion protein is aspirated off and replaced with 10 mL fresh DMEM containing 10% FBS. After 24 hours, cells receive a second dose of 500 nM TAT-hFXN fusion protein or an equal volume of vehicle in 2.5 mL DMEM medium.

Control and LRPPRC KD cells are examined for LRPPRC and COX2 levels by Western blot. Levels of LRPPRC and COX2 proteins are expected to be higher in LKPPRC KD cells treated with TAT-hFXN fusion protein when compared to untreated. Levels of SLIRP protein are expected to be modulated upon treatment with TAT-hFXN fusion protein. Levels of FXN/LRPPRC targets ADAMTS1, ATF3, CYR61, NR4a1, EGR1, EGR2, EGR3, MAOA and IFN1 are expected to be modulated by TAT-hFXN fusion protein. These results are predicted to demonstrate that TAT-hFXN fusion protein can act as a therapeutic drug for LSFC and minimize its symptoms.

### Example 5

### Treatment of LSFC-Derived Patient's Fibroblasts with TAT-hFXN Fusion Protein

LSFC patient's fibroblasts are collected from affected patients. Cells are cultured in the appropriate conditions. Cells may be maintained in high glucose DMEM medium supplemented with 10% FBS and grown to confluence. Once achieving confluency, cells are treated with 500 nM TAT-hFXN fusion protein or an equal volume of vehicle (50 mM Sodium Acetate, pH5, 1% Propylene Glycol) delivered in 2.5 mL of FBS/puromycin-free DMEM medium. After 2.5 hours, medium with TAT-hFXN fusion protein is aspirated off and replaced with 10 mL fresh DMEM containing 10% FBS. After 24 hours, cells receive a second dose of 500 nM TAT-hFXN fusion protein or an equal volume of vehicle in 2.5 mL DMEM medium. A detailed description of primary cell cultures may be found in: R Ian Freshney et al., Culture of animal cells: a manual of basic technique and specialized applications, Wiley-Blackwell, 2016.

LSFC primary culture cells are examined for LRPPRC and COX2 levels by Western blot. Levels of LRPPRC and COX2 proteins are expected to be higher in LSFC primary culture cells treated with TAT-hFXN fusion protein when compared to untreated. Levels of SLIRP protein are expected to be modulated upon treatment with TAT-hFXN fusion protein. Levels of FXN/LRPPRC targets ADAMTS1, ATF3, CYR61, NR4a1, EGR1, EGR2, EGR3, MAOA and IFN1 are expected to be modulated by TAT-hFXN fusion protein. These results are predicted to demonstrate that TAT-hFXN fusion protein can act as a therapeutic drug for LSFC and minimize its symptoms.

### Example 6

### LRPPRC Conditional Knockout (KO) Animals

A mouse model for tissue-specific LRPPRC knock out (LRPPRC-KO:Ckmm-Cre) was generated in which a NSE-Cre transgene was inserted to drive tissue specific ablation of the LRPPRC gene in the heart and in skeletal muscle (Ruzzenente et al. 2012, LRPPRC regulates mitochondrial translation. EMBO J., vol. 31, no.2, p.443-456; the entire contents of which are expressly incorporated herein by reference).

Two groups of animals (one control and one LRPPRC-KO:Ckmm-Cre) are treated with TAT-hFXN fusion protein, or vehicle (50 mM NaOAc, 0.1 PEG), via sub-cutaneous injection. The animals are treated every 48 hours. Animals are tested for cardiac performance. Post-mortem hearts are collected and mitochondrial protein and RNA (mtRNA) content are extracted and analyzed for content and for polyadenylation state.

Levels of LRPPRC and COX2 proteins are expected to be higher in the hearts of animals treated with TAT-hFXN fusion protein when compared to untreated, conditional LRPPRC-KO animals. Levels of SLIRP protein are expected to be modulated upon treatment with TAT-hFXN fusion protein. Levels of FXN/LRPPRC targets ADAMTS1, ATF3, CYR61, NR4a1, EGR1, EGR2, EGR3, MAOA and IFN1 are expected to be modulated by the TAT-hFXN fusion protein. These results are expected to show that the TAT-hFXN fusion protein can act as a therapeutic drug for LSFC and minimize its symptoms.

In addition, a recovery of the length of polyA tails, analyzed by sequencing, is expected to be observed in mtRNA from conditional LRPPRC-KO mice treated with TAT-hFXN fusion protein.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

### Example 7

### Expression of CYR61 in hFXN-KD and LRPPRC-KD Cells in Response to Treatment with TAT-hFXN Fusion Protein

The goal of this experiment was to determine changes in the levels of CYR61 in response to treatment with a TAT-hFXN fusion protein in scramble-control, LRPPRC-KD and hFXN-KD cell lines that were generated in Examples 1 and 2. To this end, scramble-control, hFXN-KD (clone A6) and LRPPRC-KD (clone KD-21C) cells were seeded on a 6-well tissue culture plate pre-coated with 1% fibronectin solution at a density of 150,000 cells/well in 1 mL of treatment media (DMEM, 5% heat inactivated FBS, 20 mM glycerol and 20 mM HEPES). After 1 hour, the cells in each well were treated with different concentrations of a TAT-hFXN fusion protein (SEQ ID NO:14). Specifically, 50 μL of a serial dilution of the TAT-hFXN fusion protein (20 5 2.5 and 1.25 as well as 0 μM control) in formulation buffer (20 mM histidine, 250 mM sucrose, 0.05% polysorbate 20, pH 5.8) was added to each well, and the plates were incubated for 3 hours in an incubator. Subsequently, 1 mL of Complete Media (10% FBS, DMEM containing antibiotics) was added to each well, and the plates were incubated for 21 hours. This cycle was repeated 3 times on days 1, 2, and 3, and then the plates were incubated for an additional day. On day 5; pictures of the plates were taken, 1 mL of media was harvested, supplemented with 10 μL HALT protease inhibitor and frozen at −80° C. for further analysis.

For CYR61 gene expression analysis, the RNA was extracted from the cells using Qiagen RNeasy Mini Kit (Cat# 74104) using the manufacturer's protocol and was quantified in duplicates using Nanodrop. The RNA was reverse transcribed using SuperScript IV VILO Master mix With ezDNAse from Invitrogen (11766050) according to the manufacturer's protocol. The cDNA was diluted to a concentration of 12.5 ng/mL. The PCR was performed in duplicates using the Advanced TAQman Mastermix and 25 ng of RNA in each PCR reaction according to the manufacturer's protocol. The TAQman Primers/Probe sets were purchased from ThermoFisher and β-actin was used as a housekeeping gene (β-actin VIC PL: Hs01060665_gl; CYR61: Hs00155479_m).

The analysis was performed by first determining the ΔCt value by subtracting the value of housekeeping gene from the value of the target gene. The ΔΔCt value was calculated by subtracting the value of vehicle-control from all other samples. Fold change was calculated using the equation Y=2^{-X} where Y is the fold change and X is the ΔΔCt value. The values for the two duplicates were averaged, and the standard deviation was calculated and graphed using a loge scale on the Y axis.

The amount of CYR61 protein secreted into the cell media was measured using the CYR61 ELISA (R&D Biosystems—CDYR10) according to the manufacturer's protocol. The media from scramble control and hFXN-KD cells was diluted 1:2 before analysis, while the media from LRPPRC-KD cells required 1:10 dilution in order to obtain results that can be plotted on the standard curve.

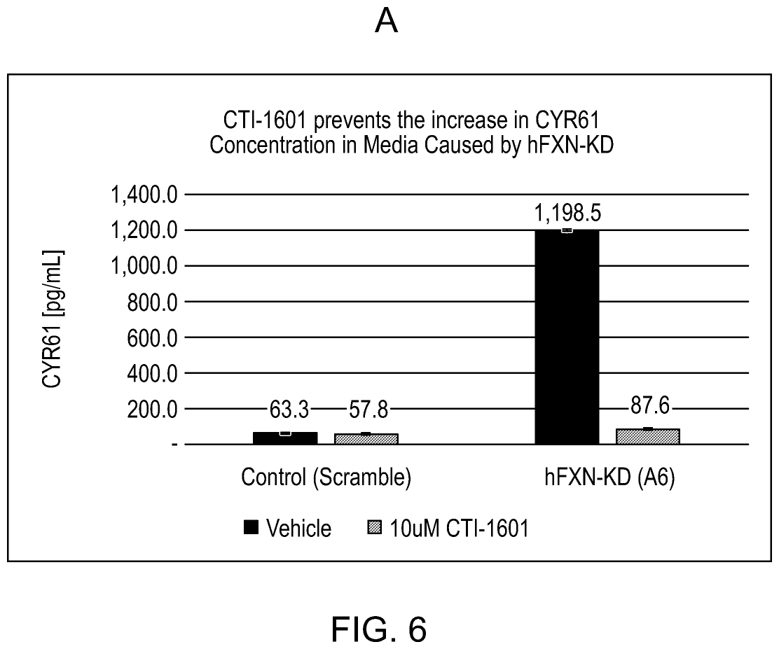

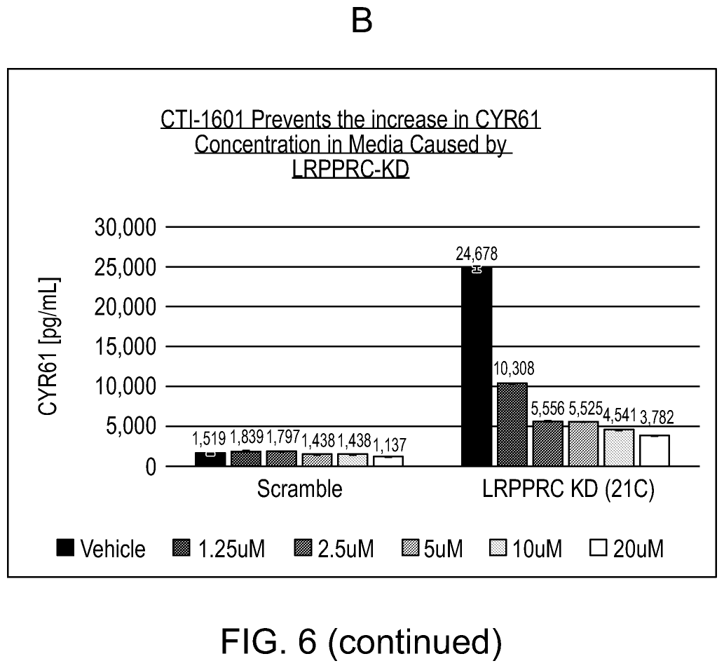

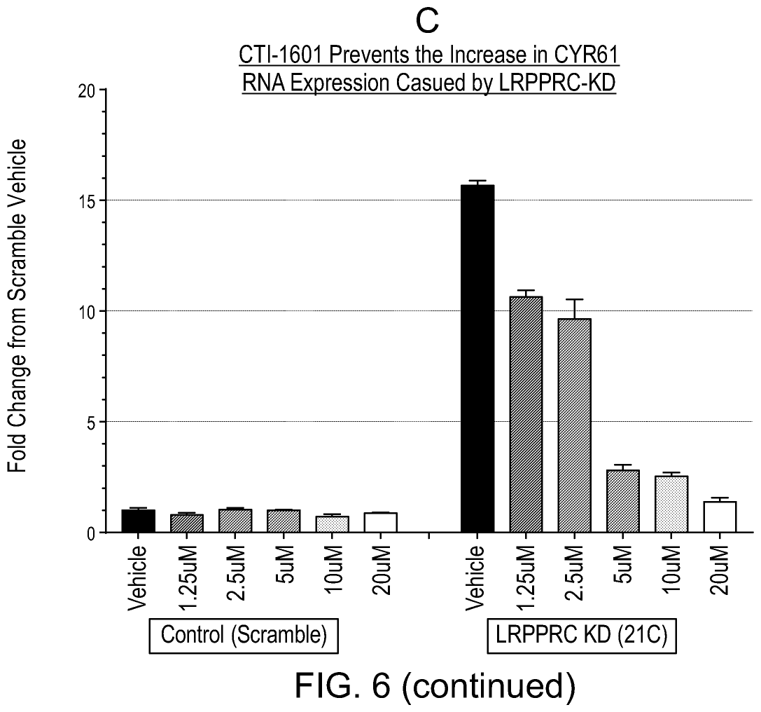

The results of the ELISA analysis for hFXN-KD cells are presented in FIG. 6, panel A. The results indicate that there is a relatively low level of CYR61 protein (about 63.3 pg/mL) in the media from the scramble control cells, and this level is not affected by treatment with 10 μM TAT-hFXN fusion protein (SEQ ID NO:14). In contrast, the levels of CYR61 protein in the media from the hFXN-KD cells that are not treated with the TAT-hFXN fusion protein (SEQ ID NO:14) are significantly higher (about 1,198.5 pg/mL) as compared to the levels in the scramble control cells. FIG. 6, panel A also indicates that the levels of the secreted CYR61 protein from the hFXN-KD cells are significantly decreased to control levels (about 87.6 pg/mL) after treatment with 20 μM TAT-hFXN fusion protein (SEQ ID NO:14).

The results of the ELISA analysis for LRPPRC-KD cells are presented in FIG. 6, panel B. The results indicate that there is a relatively low level of CYR61 protein in the media from the scramble control cells, and this level is not affected by treatment with 0-20 μM TAT-hFXN fusion protein (SEQ ID NO:14). In contrast, the levels of CYR61 protein in the media from the LRPPRC-KD cells that are not treated with the TAT-hFXN fusion protein (SEQ ID NO:14) are significantly higher (about 24,678 pg/mL) as compared to the levels in the scramble control cells (about 1,519 pg/mL). FIG. 6, panel B also indicates that the levels of the secreted CYR61 protein from the LRPPRC-KD cells decrease in response to treatment with 0-20 μM TAT-hFXN fusion protein (SEQ ID NO:14) in a dose-dependent manner.

The results of the CYR61 gene expression analysis are presented in FIG. 6, panel C. The results indicate that there is a relatively low level of CYR61 mRNA in the scramble control cells, and this level is not affected by treatment with 0-20 μM TAT-hFXN fusion protein (SEQ ID NO:14). In contrast, the levels of CYR61 mRNA in the LRPPRC-KD cells that are not treated with the TAT-hFXN fusion protein (SEQ ID NO:14) are significantly higher as compared to the levels in the scramble control cells. FIG. 6, panel B also indicates that the levels of CYR61 mRNA in the LRPPRC-KD cells decrease in response to treatment with 0-20 μM TAT-hFXN fusion protein (SEQ ID NO:14) in a dose-dependent manner.

The results of Example 7 indicate that the amount CYR61, which is a downstream target of both FXN and LRPPRC, is increased as a result of hFXN or LRPPRC deficiency, and that the increase in the amount of CYR61 may be reversed by treatment with a TAT-FXN fusion protein. Thus, CYR61 may be used as a biomarker to monitor treatment of a subject, e.g., a subject with LSFC, with FXN supplementation therapy, e.g., by administering a TAT-hFXN fusion protein, such as the specific TAT-hFXN fusion protein (SEQ ID NO:14).

### Example 8

### Treatment of LRPPRC-Deficient Cells with a TAT-hFXN Fusion Protein Prevents Acidification of Growth Media

The goal of this experiment was to determine if treatment with a TAT-hFXN fusion protein can reverse mitochondrial impairment in LRPPRC deficient cells. In this experiment, the amount of acidification of the cell culture media, e.g., due to the presence of lactic acid in the media, was used an indicator of mitochondrial function, with an increase in acidification indicating mitochondrial impairment.

Scramble control and LRPPRC KD (clone KD-21C) HEK-293 cells were seeded in at a density of 150,000 cells per well in 1 mL of treatment media (DMEM, 5% heat inactivated FBS, 20 mM glycerol, and 20 mM HEPES) on a 6-well tissue culture plate pre-coated with 1% fibronectin solution. After 1 hour, the cells in each well were treated with different concentrations of a TAT-hFXN fusion protein (SEQ ID NO:14). Specifically, 50 μL of a serial dilution of the TAT-hFXN fusion protein (SEQ ID NO:14) (20 μM, 10 μM, 5 μM, 2.5 μM, and 1.25 μM, as well as 0 μM control) in formulation buffer (20 mM histidine, 250 mM sucrose, 0.05% polysorbate 20, pH 5.8) was added to each well, and the plates were incubated for 3 hours in an incubator. Subsequently, 1 mL of Complete Media (10% FBS, DMEM containing antibiotics) was added to each well, and the plates were incubated for 21 hours. This cycle was repeated 3 times on days 1, 2, and 3, and then the plates were incubated for an additional day. On day 5; pictures of the plates were taken, 1 mL of media was harvested, supplemented with 10 μL HALT protease inhibitor and frozen at −80° C. for further analysis. The RNA was extracted from the cells using Qiagen RNeasy Mini Kit (Cat# 74104) using the manufacturer's protocol.

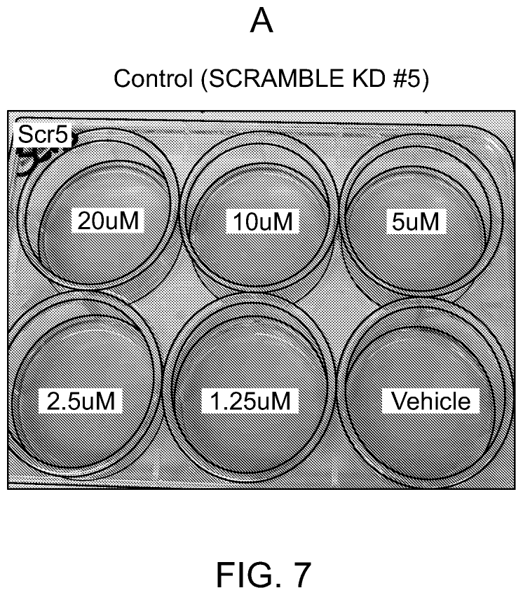

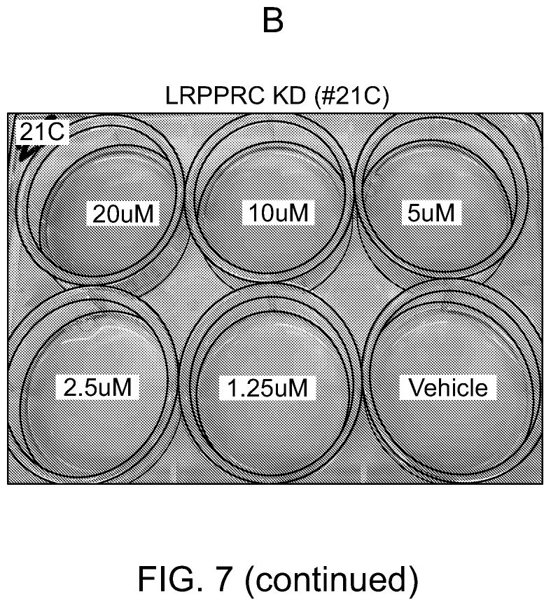

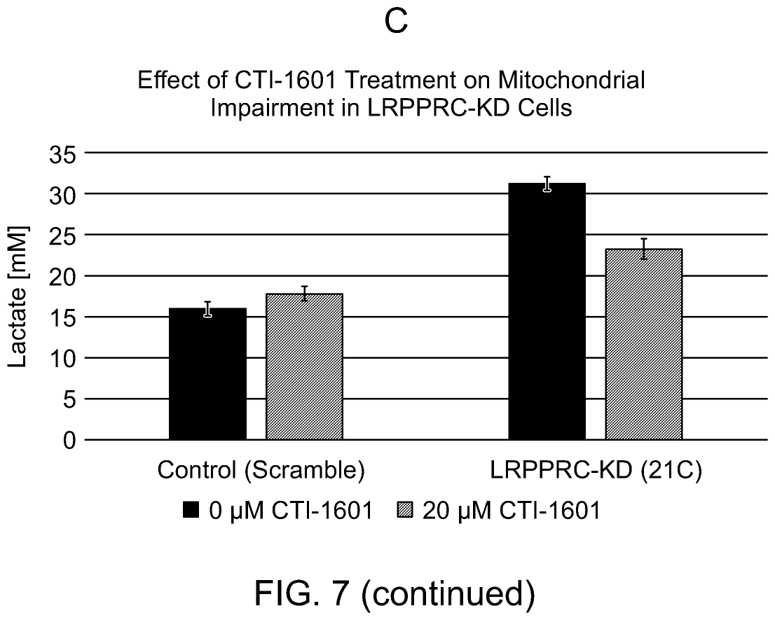

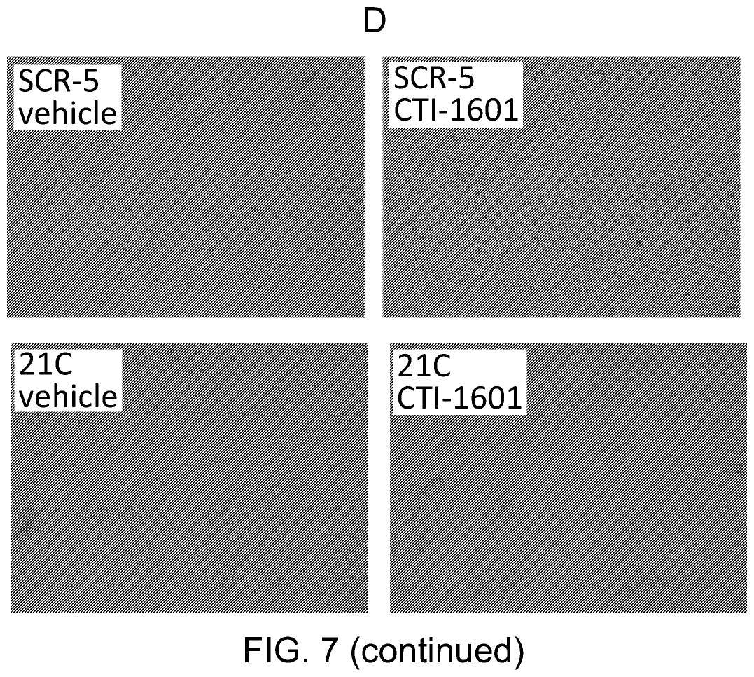

Phenol Red is the pH indicator used in DMEM media. As shown in FIG. 7, panel A, the scramble control cells did not cause a significant change in the pH of the media. In contrast, as shown in FIG. 7, panel B, the LRPPRC-KD cells caused the media to turn yellow (vehicle well), indicating acidification of the media. As also shown in FIG. 7, panel B, treatment of the LRPPRC KD cells with increasing concentrations of the TAT-hFXN fusion protein (SEQ ID NO:14) prevented the acidification of the media (1.25 uM and 2.5 uM wells contains yellow media; 5 uM and 10 uM wells contain orange media; and 20 uM well contain red media) in a dose-dependent manner. The results shown in FIG. 7 indicate that a TAT-hFXN fusion protein (e.g., SEQ ID NO:14) can reverse the effect of mitochondrial impairment induced by LRPPRC-KD.

Levels of lactate were measured in the media from the scramble control cells and LRPPRC KD cells using Lactate Glo Assay (Promega J5021) according to the manufacturer's protocol. The media was diluted 1:500 on a white opaque 96 well plate (Corning 3917). As shown in FIG. 7, panel C, growing LRPPRC-KD cells cause a significant increase in the amount of lactic acid in the media as compared to the scramble control cells. Treatment of the scramble control cells with 20 μM TAT-hFXN fusion protein (SEQ ID NO:14) had no significant effect on the amount of lactic acid in the media. However, treatment of LRPPRC KD cells with 20 μM TAT-hFXN fusion protein (SEQ ID NO:14) caused a significant 25% reduction of the levels of lactic acid in the media.

Cell viability and density were assessed visually using microscopic images. As shown in FIG. 7, panel D, no change in viability or density was observed for scramble control and LRPPRC-KD cells following treatment with 20 μM TAT-hFXN fusion protein (SEQ ID NO:14) or vehicle control.

The results of Example 8 indicate that LRPPRC-KD cells are characterized by a mitochondrial impairment as indicated by acidification of the cell culture media, and that this impairment is reversed in a dose-dependent manner by treatment with a TAT-hFXN fusion protein.

### Example 9

### Transfection of hFXN into hFXN-KD and LRPPRC-KD Cells Causes a Decrease in the Amount of Secreted CYR61 Protein

The goal of this experiment was to determine if transfection of hFXN-KD and LRPPRC-KD cells with hFXN can reverse the mitochondrial impairment in these cells as measured by the amount of secreted CYR61 protein. To this end, scramble KD, hFXN-KD and LRPPRC-KD cells were transfected with an empty pCDNA3 vector (+V) or with an expression vector for full-length hFXN: pCDNA3-hFXN (+hFXN) using Fugene-6 reagent according to the manufacturer's instructions and incubated for 48 hours. The transfected cells were incubated for additional 48 hours. After the second 48-hour incubation, 1 mL of media was removed and 10 μL of HALT protease inhibitor was added to the aliquot. The amount of CYR61 protein in the media was measured using Simple Step CYR61 Elisa from ABCAM (ab238267) according to the manufacturer's protocol. For the measurement, the media from the LRPPRC-KD cells was diluted 1/100, while the media from Scramble and hFXN-KD were diluted 1/10. Data was plotted using Graphpad Prism Bar graph with Standard Deviation as error bars.

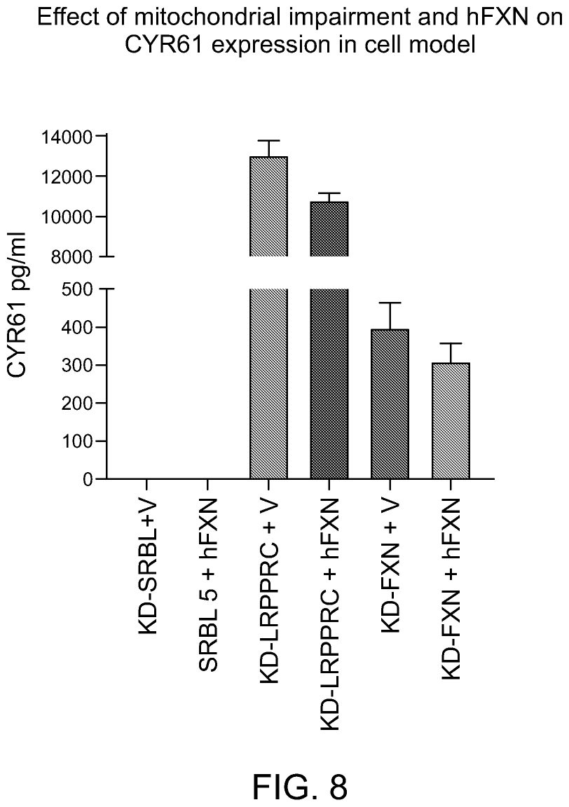

The results are shown in FIG. 8. FIG. 8 indicates that the scramble control cells do not secrete detectable levels of CYR61 protein either in the absence or in the presence of exogenously expressed hFXN. There is abundant CYR61 protein secreted from the hFXN-KD and LRPPRC-KD cells transfected with an empty vector, and the amount of secreted CYR61 protein is decreased by the transient expression of hFXN in these cells.

The results of Experiment 9 indicate that CYR61 may be used as a biomarker to monitor treatment of a subject, e.g., a subject with LSFC, with frataxin supplementation therapy, e.g., by administering a TAT-hFXN fusion protein, such as the TAT-hFXN fusion protein (SEQ ID NO:14).

### Example 10

### Production of FNX-Knockout Mouse Embryonic Stem Cell Line

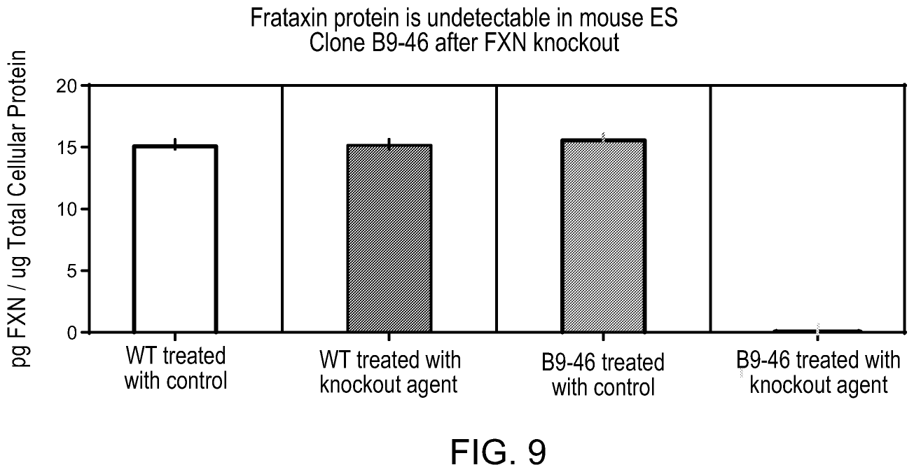

The goal of this experiment was to produce a mouse embryonic stem (ES) cell line deficient in frataxin. A homozygous mouse ES clone B9-46, which was produced as a result of this experiment, may be induced to knock out both alleles of the frataxin gene. FIG. 9 is a bar graph showing the amount of frataxin protein per total cellular protein in the WT mouse ES clone and the homozygous mouse ES clone B9-46 which have been treated with an agent to induce the frataxin knockout or a control. The amount of mouse frataxin protein was measured using Mouse FXN Elisa kit (Abcam ab199078) according to manufacturer's protocol. FIG. 9 indicates that treatment with an agent to induce frataxin knockout resulted in the elimination of the frataxin protein in B9-46 cells. No decrease in the levels of the frataxin protein was observed in the WT cells or control-treated B9-46 cells.

### Example 11

### Level of CYR61 is Increased in the FXN Knockout Mouse ES Cells

The goal of this experiment was to determine if levels of the secreted CYR61 protein are altered in the mouse ES B9 cells with the FXN gene knocked out. To this end, mouse B9 cells were treated with an agent to induce knockdown of the FXN gene or a control.

To measure the amount of CYR61 gene expression, RNA was extracted from the B9 mouse cells and the amount of CYR61 mRNA was measured using qPCR as previously described. The TaqMan Primers used for the qPCR analysis were purchased from ThermoFisher and β-actin was used as a housekeeping gene (β-actin VIC PL: Hs01060665_gl; CYR61: Hs00155479_ml). Two biological replicates were analyzed for each of agent and control treatment.

To measure the amount of secreted CYR61 in cell media, 1 mL of cell media was harvested, supplemented with 10 μL HALT protease inhibitor and frozen at −80° C. for further analysis. The amount of the secreted CYR61 protein was measured using ELISA as previously described.

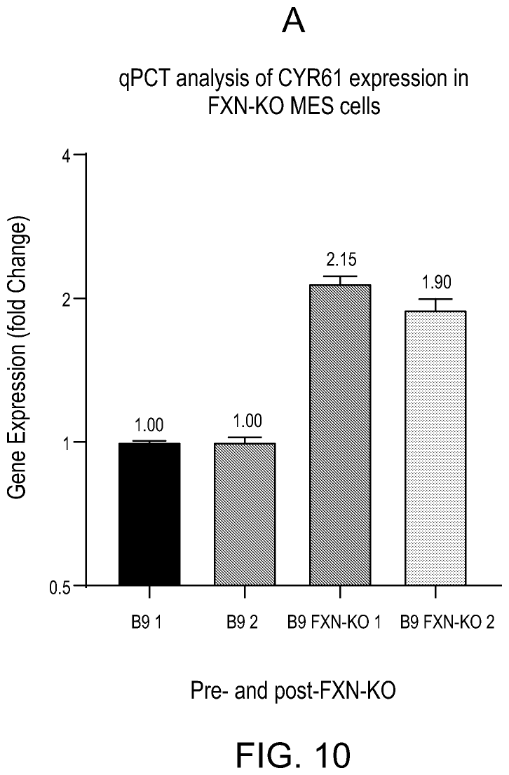

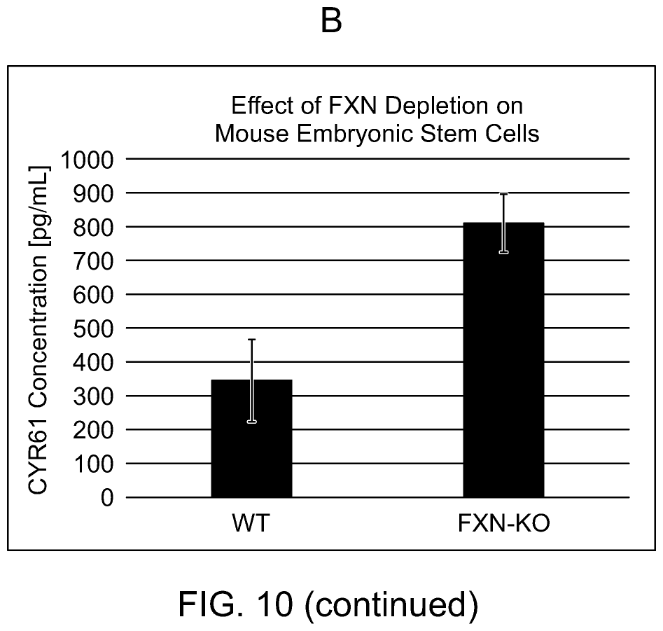

The results of the CYR61 gene expression analysis are presented in FIG. 10, panel A. The results indicate that knockout of the FXN gene in the B9 cells results in an approximate 2-fold increase in the expression of CYR61 mRNA. The results of the measurement of the levels of secreted CYR61 protein are presented in FIG. 10, panel B. The results indicate that knockout of the FXN gene in the B9 cells results in an approximate 2-fold increase in the amount of CYR61 protein levels in the cell media.

Descriptions of embodiments of the disclosure in the present application are provided by way of example and are not intended to limit the scope of the disclosure. The described embodiments comprise different features, not all of which are required in all embodiments. Some embodiments utilize only some of the features or possible combinations of the features. Variations of embodiments of the disclosure that are described, and embodiments comprising different combinations of features noted in the described embodiments, will occur to persons of the art. The scope of the disclosure is limited only by the claims.

What is claimed is:

1. A method of treating Leigh Syndrome, French Canadian Type (LSFC), said method comprising administering to a subject in need thereof an effective amount of a frataxin (FXN) therapeutic compound, such that said LSFC in said subject is treated.

2. A method of modulating LRPPRC in a subject affected by Leigh Syndrome, French Canadian Type (LSFC), said method comprising administering to said subject an effective amount of a frataxin (FXN) therapeutic compound, such that said LRPPRC in said subject is modulated.

3. A method of modulating SLIRP in a subject affected by Leigh Syndrome, French Canadian Type (LSFC), said method comprising administering to said subject an effective amount of a frataxin (FXN) therapeutic compound, such that said SLIRP in said subject is modulated.

4. The method of any one of claims 1-3, wherein the level of at least one downstream target of FXN and/or LRPPRC is modulated in said subject.

5. The method of claim 4, wherein said downstream target of FXN and/or LRPPRC is selected from the group consisting of ADAMTS1, ATF3, CYR61, NR4a1, EGR1, EGR2, EGR3, MAOA and IFN 1.

6. The method of any one of claims 1-5, wherein mitochondrial RNA is stabilized in said subject.

7. A method of modulating LRPPRC in a cell, said method comprising contacting said cell with an effective amount of a frataxin (FXN) therapeutic compound, such that said LRPPRC in said cell is modulated.

8. A method of modulating SLIRP in a cell, said method comprising contacting said cell with an effective amount of a frataxin (FXN) therapeutic compound, such that said SLIRP in said cell is modulated.

9. The method of claim 7 or claim 8, wherein the level of at least one downstream target of FXN and/or LRPPRC is modulated in said cell.

10. The method of claim 9, wherein said downstream target of FXN and/or LRPPRC is selected from the group consisting of ADAMTS1, ATF3, CYR61, NR4a1, EGR1, EGR2, EGR3, MAOA and IFN 1.

11. The method of any one of claims 7-10, wherein said cell is in a subject.

12. The method of claim 11, wherein said subject is affected by LSFC.

13. The method of any one of claims 1-12, wherein said frataxin therapeutic compound comprises a polypeptide comprising frataxin, or a fragment, variant or derivative thereof

14. The method of any one of claims 1-12, wherein said frataxin therapeutic compound comprises a nucleic acid sequence encoding a polypeptide comprising frataxin, or a variant, fragment or derivative thereof.

15. The method of claim 13, wherein said frataxin therapeutic compound comprises a fusion protein comprising frataxin, or a variant, fragment or derivative thereof, and an at least one different amino acid sequence.

16. The method of claim 15, wherein said fusion protein comprises an amino acid sequence with at least about 85% sequence identity to SEQ ID NO: 1 or SEQ ID NO: 2, or a fragment or derivative thereof.

17. The method of claim 16, wherein said fusion protein comprises an amino acid sequence with at least about 90%, at least about 95% or at least about 99% sequence identity to SEQ ID NO: 1 or SEQ ID NO: 2, or a fragment or derivative thereof.

18. The method of claim 17, wherein said fusion protein comprises an amino acid sequence of SEQ ID NO: 1, SEQ ID NO: 2, or a fragment or derivative thereof.

19. The method of any one of claims 15-18, wherein said at least one different amino acid sequence comprises a cell penetrating peptide (CPP).

20. The method of claim 19, wherein the CPP comprises a peptide selected from the group of CPPs listed in the Database of Cell-Penetrating Peptides CPPsite 2.0, or a variant, fragment or derivative thereof.

21. The method of claim 19, wherein the CPP comprises an amino acid sequence with at least about 85%, at least about 90%, at least about 95% or at least about 99% sequence identity to any one of SEQ ID NOs. 4-13, or a fragment or derivative thereof.

22. The method of claim 20, wherein said CPP comprises an amino acid sequence with at least about 85%, at least about 90%, at least about 95% or at least about 99% sequence identity to the transduction domain of HIV-TAT (SEQ ID NO: 4), or a fragment or derivative thereof

23. The method of claim 22, wherein said CPP comprises the amino acid sequence of the transduction domain of HIV-TAT (SEQ ID NO: 4), or a fragment or derivative thereof.

24. The method of any one of claims 1-23, wherein said frataxin therapeutic compound comprises a fusion protein comprising an amino acid sequence with at least about 85% sequence identity to SEQ ID NO: 14, or a fragment or derivative thereof

25. The method of claim 24, wherein said frataxin therapeutic compound comprises a fusion protein comprising an amino acid sequence with at least about 90%, at least about 95% or at least about 99% sequence identity to SEQ ID NO: 14, or a fragment or derivative thereof

26. The method of any one of claims 1-25, wherein said frataxin therapeutic compound comprises a fusion protein comprising the amino acid sequence of SEQ ID NO: 14, or a fragment or derivative thereof.

27. A method of treating Leigh Syndrome, French Canadian Type (LSFC), said method comprising administering to a subject in need thereof an effective amount of a frataxin (FXN) therapeutic compound,
wherein said frataxin therapeutic compound comprises a fusion protein comprising the amino acid sequence of SEQ ID NO: 14, or a variant, fragment or derivative thereof, such that said LSFC in said subject is treated.

28. A method for evaluating effectiveness of frataxin (FXN) supplementation therapy in a subject with LSFC, the method comprising:
(a) determining a level of CYR61 in a sample from said subject following treatment with FXN supplementation therapy;
(b) comparing the level of CYR61 in (a) with a baseline CYR61 level; and
(c) using the comparison in (b) to determine effectiveness of the FXN supplementation therapy in said subject.

29. The method of claim 28, further comprising determining a baseline CYR61 level in a sample from a subject with LSFC obtained prior to administration of the FXN supplementation therapy.

30. The method according to claim 28 or 29, wherein determining a level of CYR61 comprises determining the level of CYR61 protein in the sample.

31. The method according to claim 28 or 29, wherein determining a level of CYR61 comprises determining the level of CYR61 mRNA in the sample.

32. A method of detecting CYR61 in a biological sample from a subject with Leigh Syndrome, French Canadian Type (LSFC), comprising contacting the biological sample, or a portion thereof, with one or more detection reagents specific for detection of CYR61.

33. The method of claim 32, wherein the subject is being treated with a frataxin supplementation therapy.

34. The method of any one of claims 28-33, wherein the frataxin supplementation therapy comprises treating the subject with a frataxin fusion protein.

35. The method of claim 34, wherein said fusion protein comprises an amino acid sequence with at least about 85% sequence identity to SEQ ID NO: 14, or a fragment or derivative thereof.

36. The method of claim 35, wherein said fusion protein comprises an amino acid sequence with at least about 90%, at least about 95% or at least about 99% sequence identity to SEQ ID NO: 14, or a fragment or derivative thereof.

37. The method claim 36, wherein said fusion protein comprises the amino acid sequence of SEQ ID NO: 14, or a fragment or derivative thereof.

38. A method of treating lactic acidosis in a subject with Leigh Syndrome, French Canadian Type (LSFC), said method comprising administering to said subject an effective amount of a frataxin (FXN) therapeutic compound, such that said lactic acidosis in said subject is treated.

39. The method of claim 38, wherein said frataxin therapeutic compound comprises a 7polypeptide comprising frataxin, or a fragment, variant or derivative thereof.

40. The method of claim 38, wherein said frataxin therapeutic compound comprises a nucleic acid sequence encoding a polypeptide comprising frataxin, or a variant, fragment or derivative thereof.

41. The method of claim 40, wherein said frataxin therapeutic compound comprises a fusion protein comprising frataxin, or a variant, fragment or derivative thereof, and an at least one different amino acid sequence.

42. The method of claim 41, wherein said fusion protein comprises an amino acid sequence with at least about 85% sequence identity to SEQ ID NO: 1 or SEQ ID NO: 2, or a fragment or derivative thereof.

43. The method of claim 42, wherein said fusion protein comprises an amino acid sequence with at least about 90%, at least about 95% or at least about 99% sequence identity to SEQ ID NO: 1 or SEQ ID NO: 2, or a fragment or derivative thereof.

44. The method of claim 43, wherein said fusion protein comprises an amino acid sequence of SEQ ID NO: 1, SEQ ID NO: 2, or a fragment or derivative thereof.

45. The method of any one of claims 41-44, wherein said at least one different amino acid sequence comprises a cell penetrating peptide (CPP).

46. The method of claim 45, wherein the CPP comprises a peptide selected from the group of CPPs listed in the Database of Cell-Penetrating Peptides CPPsite 2.0, or a variant, fragment or derivative thereof.

47. The method of claim 45, wherein the CPP comprises an amino acid sequence with at least about 85%, at least about 90%, at least about 95% or at least about 99% sequence identity to any one of SEQ ID NOs. 4-13, or a fragment or derivative thereof.

48. The method of claim 47, wherein the CPP comprises an amino acid sequence with at least about 85%, at least about 90%, at least about 95% or at least about 99% sequence identity to the transduction domain of HIV-TAT (SEQ ID NO: 4), or a fragment or derivative thereof.

49. The method of claim 48, wherein the CPP comprises the amino acid sequence of the transduction domain of HIV-TAT (SEQ ID NO: 4), or a fragment or derivative thereof.

50. The method of any one of claims 38-49, wherein said frataxin therapeutic compound comprises a fusion protein comprising an amino acid sequence with at least about 85% sequence identity to SEQ ID NO: 14, or a fragment or derivative thereof.

51. The method of claim 50, wherein said frataxin therapeutic compound comprises a fusion protein comprising an amino acid sequence with at least about 90%, at least about 95% or at least about 99% sequence identity to SEQ ID NO: 14, or a fragment or derivative thereof.

52. The method of claim 51, wherein said frataxin therapeutic compound comprises a fusion protein comprising the amino acid sequence of SEQ ID NO: 14, or a fragment or derivative thereof.

53. A method of treating lactic acidosis in a subject with Leigh Syndrome, French Canadian Type (LSFC), said method comprising administering to said subject an effective amount of a frataxin (FXN) therapeutic compound,
wherein said frataxin therapeutic compound comprises a fusion protein comprising the amino acid sequence of SEQ ID NO: 14, or a variant, fragment or derivative thereof, such that said lactic acidosis in said subject is treated.
